(19) United States
(12) Patent Application Publication
(10) Pub. No.: US 2025/0308677 A1
(43) Pub. Date: Oct. 2, 2025

(54) SYSTEM AND METHOD FOR MANAGING ELECTRONIC MEDICAL RECORDS AND MEDICAL PRACTICES

(71) Applicant: POCKET MD CORP., Ottawa (CA)

(72) Inventor: Jeevan Jot Singh, Ottawa (CA)

(21) Appl. No.: 18/620,647

(22) Filed: Mar. 28, 2024

(51) Int. Cl. G16H 40/20; G06Q 40/08; G16H 20/10; G16H 80/00

(52) U.S. Cl. CPC G16H 40/20; G06Q 40/08; G16H 20/10; G16H 80/00

(57) ABSTRACT

A system and method for managing a medical practice. The system includes a communications interface configured to receive data from a first and a second client terminal. The system also includes a database of patient medical records. The system further includes a processor configured to schedule patient appointments requested from the first client terminal and receive medical data from a second client terminal. The processor is also configured to store the received medical data in the database of medical records. If a medical test is needed based on the received medical data, the processor is configured to generate a request for the medical test, send the request for the medical test to a medical test provider, and receive a test result from the medical test provider. If a prescription is needed, the processor is configured to generate the prescription, and send the prescription electronically to a pharmacy.

### FIELD OF INVENTION

The present invention relates generally to a system and method for managing electronic medical records and a medical practice.

### BACKGROUND OF THE INVENTION

Physicians and clinics are increasingly burdened with scheduling, billing, and the tracking of medical records. To that end, many physicians limit the number of patients that they can intake and see as with each patient, there is an increase in administrative work. In many instances, this is due to a lack of coordination and efficiency in using different tools to manage a medical practice. Prior art has indicated that the fragmentation of using different tools leads to physicians spending 66 minutes a day flipping through screens, and spending half of their day on desk work with several hours of unpaid time, ultimately leading to 79% burnout.

Additionally, physicians currently work in a siloed approach, where a physician treats patients individually, and where if another physician or medical practitioner were required to see the patient, they would have no visibility of the patient's medical history and would have to actively obtain said patient's medical history.

Furthermore from the patient's point of view, if there is ever a need to switch physicians, or the need to visit more than one medical practitioner, there is a need to do a new intake each time, in order to provide the patient's medical history. This also provides low flexibility for patients to move between medical practitioners, as there is a view that moving between medical practitioners may be more hassle than it is worth.

### SUMMARY OF INVENTION

According to various aspects of the present invention, there is provided a system for managing a medical practice. The system includes a communications interface connected to a network, wherein the communications interface is configured to receive data from a first client terminal and a second client terminal. The system further includes a memory storage unit. The memory storage unit includes a database of patient medical records. The system also includes a processor connected to the communications interface and the memory storage unit. The processor is configured to schedule patient appointments requested from the first client terminal and receive medical data from a second client terminal. The processor is also configured to store the medical data in the database of medical records. If a medical test is needed based on the received medical data, the processor is configured to generate a request for the medical test, send the request for the medical test to a medical test provider, and receive a test result from the medical test provider. If a prescription is needed based on the received medical data, the processor is further configured to generate the prescription, and send the prescription electronically to a pharmacy.

In one embodiment, the processor may be configured to generate an insurance claim and send the insurance claim to a medical insurance provider.

In another embodiment, the memory storage unit further includes an artificial intelligence engine trained on the database of medical records. The processor may be further configured to provide suggestions of whether a medical test or prescription is needed based the received medical data using the artificial intelligence engine.

In yet another embodiment, the memory storage unit further includes a database of physician information. Additionally, the processor configured to generate the prescription includes receiving a medication to be prescribed from the second client terminal, querying the database of medical records to retrieve the patient's name, querying the database of physician information to retrieve a physician's name and a physician's signature, and assembling the prescription, the prescription including at least the medication to be prescribed, the patient's name, the physician's name and the physician's signature.

In another embodiment, the communications interface may further be configured to receive data from a validation server, where the database of patient medical records includes patient insurance data. The processor may further be configured to verify the patient insurance data against the validation server prior scheduling patient appointments requested from the first client terminal.

According to various aspects of the present invention, there is provided a method of administering a medical appointment. The method includes scheduling the medical appointment from a first client terminal, receiving patient medical data from a second client terminal and storing the received patient medical data in a database of medical records. The method further includes determining whether a medical test is needed based on the received medical data. If the medical test is needed, the method includes generating a request for the medical test, sending the request for the medical test to the first client terminal, and receiving a test result from a medical test provider. The method also includes determining whether a prescription is needed based on the received medical data. If the prescription is needed, the method includes generating the prescription, and sending the prescription electronically to a pharmacy.

In one embodiment, the method may also include generating an insurance claim and sending the insurance claim to a medical insurance provider.

In another embodiment, the method may also include providing suggestions of whether the medical test or prescription is needed based on the received medical data using an artificial intelligence engine trained on the database of medical records.

In yet another embodiment, generating the prescription may also include receiving a medication to be prescribed from the second client terminal, querying the database of medical records to retrieve the patient's name, querying the database of physician information to retrieve a physician's name and a physician's signature, and assembling the prescription, the prescription including at least the medication to be prescribed, the patient's name, the physician's name and the physician's signature.

In another embodiment, the method may further include receiving patient insurance data from the first client terminal, and verifying the patient insurance data against a validation server prior to scheduling patient appointments requested from the first client terminal.

### BRIEF DESCRIPTION OF THE DRAWINGS

The embodiments of the present invention shall be more clearly understood with reference to the following detailed description of the embodiments of the invention taken in conjunction with the accompanying drawings, in which:

FIG. 1 depicts an example system for managing electronic medical records and for managing a medical practice;

FIG. 2 depicts an alternate example system for managing electronic medical records and for managing a medical practice;

FIG. 3 depicts an example method for managing the electronic medical record of a patient in accordance with the example system of FIG. 1;

FIG. 4 depicts a continuation of the example method of FIG. 3;

FIG. 5 depicts an example method for ordering tests in accordance with the example system of FIG. 1;

FIG. 6 depicts an example method for creating prescriptions in accordance with the example system of FIG. 1;

FIG. 7 depicts an example method for generating a bill for the patient in accordance with the example system of FIG. 1;

FIG. 8 to FIG. 45 depicts example graphical user interfaces of the example system of FIG. 1;

FIG. 46 depicts another example system for managing electronic medical records and for managing a medical practice;

FIG. 47 depicts another example method for managing the electronic medical records of patients in accordance with the example system of FIG. 47;

FIG. 48 depicts an example method of referring a patient to another medical professional or medical specialist; and

FIG. 49 to FIG. 56 depicts example graphical user interfaces of the example system of FIG. 47.

### DESCRIPTION OF THE PREFERRED EMBODIMENTS OF THE INVENTION

The description, which follows, and the embodiments described therein are provided by way of illustration of an example, or examples of particular embodiments of principles and aspects of the present invention. These examples are provided for the purposes of explanation and not of limitation, of those principles of the invention. In the description that follows, like parts are marked throughout the specification and the drawings with the same respective reference numerals.

By way of general overview, there is provided a system and method of managing a medical practice and a patient's electronic medical records. In at least some embodiments, the advantage of the system described below allows flexibility for patients to move between physicians, and also allows patients ease of access in looking at their own medical records. Furthermore, the system also provides lower overhead in cost and labor for physicians when managing a medical practice, allowing more time to be spent with patients, or additional time to intake new patients.

Specifically, system 100 includes server 104 in communication over WAN 128 with patient client terminal 120 and physician client terminal 124. Patient profiles may be created, where data pertaining to the patient may be verified against validation databases 132 (also referred to herein as validation server 132), and stored in patient database 136. Physician profiles may also be created, where date may be stored in physician database 140. Medical appointments may be scheduled, may occur virtually. During the virtual medical appointments, appointment date and patient data, including the patient's vitals, any medical tests that are required and any medications that need to be prescribed may be provided to server 104 concurrently, saving time for physicians, and ensuring accuracy of data as it is updated live and in the moment. Server 104 may also perform other services, including, but not limited to, generating and providing all the necessary documentation for ordering medical tests to a patient, generating prescriptions for the patient and sending said prescriptions to the pharmacy, and billing the client.

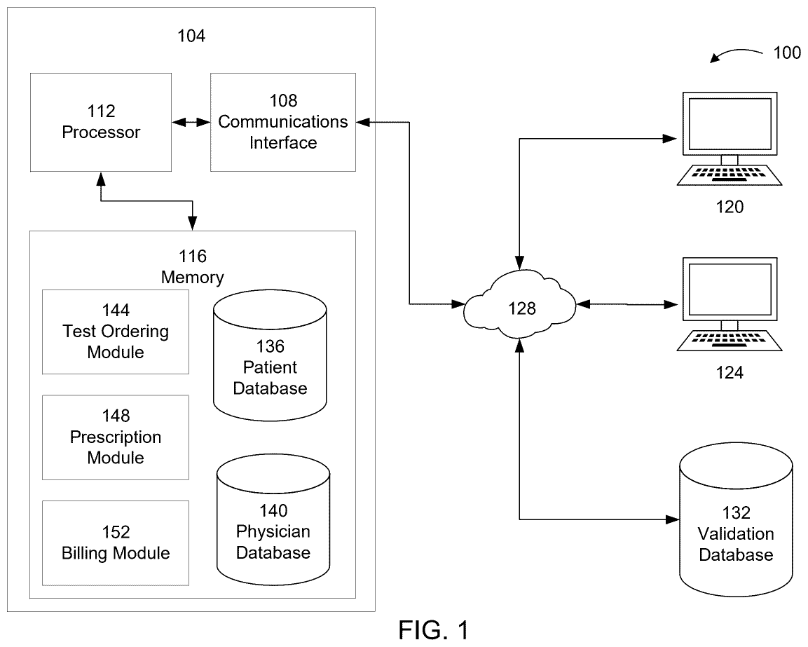

FIG. 1 depicts a system 100 for managing electronic medical records of a patient and the medical practice of a physician. Specifically, system 100 includes server 104 communicating with patient client terminal 120, physician client terminal 124, and validation database 132 via wireless access network (“WAN”) 128. More specifically, server 104 includes communications interface 108, processor 112 and memory 116, where patient profiles with associated medical data may be created and stored, virtual appointments between patients and physicians may be scheduled and hosted, medical tests may be ordered, prescriptions may be generated, and bills may be sent from the physician to the patients Components of system 100 will be discussed further in detail below.

Server 104 includes a processor 112 interconnecting a memory 116 and a communications interface 108. The processor can include a central-processing unit (CPU), a graphics processing unit (GPU), a microcontroller, a microprocessor, a processing core, a field-programmable gate array (FPGA), or similar. In some embodiments, the processor 112 can include multiple cooperating processors. The processor 112 can cooperate with non-transitory computer readable medium, such as the memory 116 to execute instructions to realize the functionality discussed herein.

Memory 116 can include a combination of volatile memory (e.g. Random Access Memory or RAM) and non-volatile memory (e.g. non-volatile random-access memory, read only memory or ROM, Electrically Erasable Programmable Read Only memory or EEPROM, flash memory). All or some of the memory 116 can be integrated with processor 112. Memory 116 stores computer reasonable instructions for execution by processor 112.

It will now be apparent that each element of memory 116 can be carried out by the processor 112 executing operations. In other words, functionality described below as being carried out by a module of memory 116 or a module of server 104 can be based on any known server environment.

In some embodiments, memory 116 stores a plurality of computer-readable data and programming instructions, accessible by processor 112, in the form of software objects, such as various applications, queries or types of data for use during the execution of those applications. In particular, the execution of the instructions in memory 116 by processor 112 allow for the creation of patient profiles, and the verification of patient profile data to be stored in patient database 136. In addition, the execution of instructions and memory 116 also allows for the creation of physician profiles to be stored in physician database 140. Furthermore, the execution of instructions in memory 116 by processor 112 queries or request data from the patient database 136 and the physician database 140 to order medical tests, create prescriptions, and generate bills. The person skilled in the art will now recognize the various forms of computer readable programming instructions stored in memory 116 that can be executed by processor 112 as applications or queries.

In at least some embodiments, memory 116 stores patient database 136. Patient database 136 includes at least bibliographic information about a patient, such as the patient's name, date of birth and address. Patient database 136 may also include the pharmacy of choice for a patient to pick up their medication. Another embodiments patient database 136 may also include payment methods to pay medical bills and emergency contacts. Furthermore, in at least some embodiments, patient database, 136 will include data pertaining to health cards, such as a health card number. Data pertaining to health cards may be used to verify a patient's identity against validation database 132. A person skilled in the art will recognize the different information that may be stored in patient database. 136.

In at least some embodiments, memory 116 stores physician database 140. Physician database 140 includes at least the biographic information about a physician, including but not limited to, the physician's practicing province, the physician's license number, and the physician's medical liability information. Physician database 140 may also include uploaded documents, such as proof of insurance, proof of identification and proof of license, as validation of the physician being licensed to practice medicine. Furthermore, physician database 140 may also include the signature of the doctor, for use in prescriptions. In alternate embodiments, the data stored in physician database 140 associated with each physician may be verified against external databases (not shown). A person skilled in the art will recognize the different information that may be stored in physician database 140.

In at least some embodiments, memory 116 stores test ordering module 144. The execution of test ordering module 144 by processor 112 allows for the ordering of medical tests, including, but not limited to, an MRI requisition, a HIV serology, a general test requisition, a public health test, an imaging requisition, providing a general note, and providing a referral letter for a medical test. The test ordering module 144 provides a graphical user interface for the physician on physician client terminal 124 to enter information pertaining to the relevant test for the patient, and generates a medical test request form to be sent to the patient terminal 120, providing the patient with the medical test request form to take to a lab or a testing facility. The test ordering module 144 further receives test results from the lab or testing facility, logs the test results on patient database 136 and provides the test results for the physician on the physician client terminal 124 to review. A person skilled in the art will recognize the different functions of test ordering module 144.

In at least some embodiments, memory 116 stores prescription module 148. The execution of prescription module 148 by processor 112 allows for the generation and sending of prescriptions. In the current embodiment, prescription module 148 provides a graphical user interface for the physician on physician client terminal 124 to input medications that a patient may require. Furthermore, prescription module 148 generates the prescription as according to standard medical practices, and sends the prescription to the relevant pharmacy to be fulfilled. Prescription module 148 also logs the prescription. A person skilled in the art will recognize the different functions of prescription module 148.

In at least some embodiments, memory 116 stores billing module 152. The execution of billing module 152 allows the generation of a bill based on the medical services provided by the physician towards a patient. Billing module 152 further provides a graphical user interface on the physician terminal 124 for the physician to review and edit as necessary. Billing module 152 then sends the bill to the patient, and logs a copy of the bill on patient database 136. A person skilled in the art will recognize the different functions of billing module 152.

Server 104 further includes communications interface 108. Communications interface 108 allows for processor 112 to communicate with WAN 128. Communications interface 108 includes suitable hardware (e.g. transmitters, receivers, network interface controllers and the like) allowing server 104 to communicate with other components in system 100, such as patient client terminal 120, physician client terminal 124 and validation database 132. The specific components of communications interface 108 may be selected based on the type of network or other links server 104 may be required to communicate over.

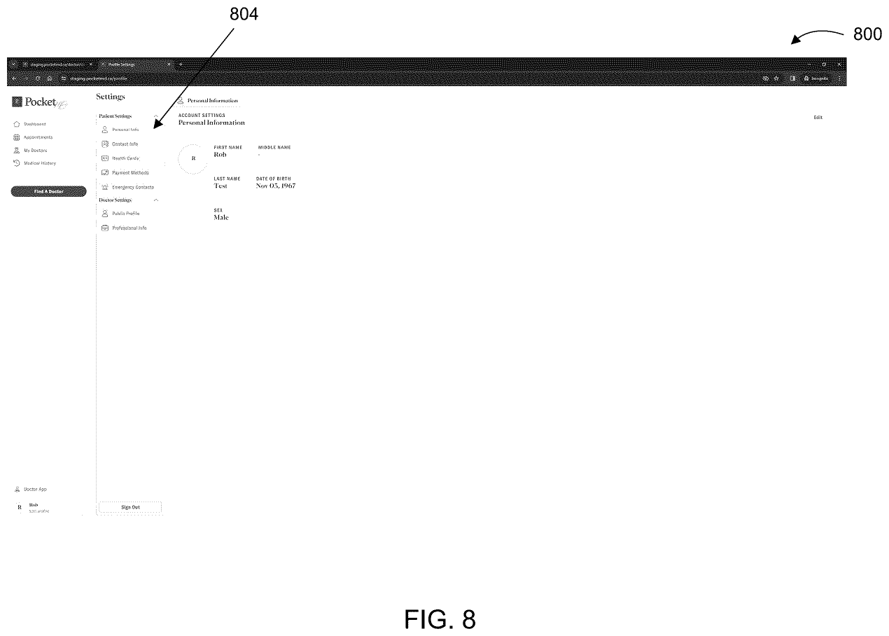

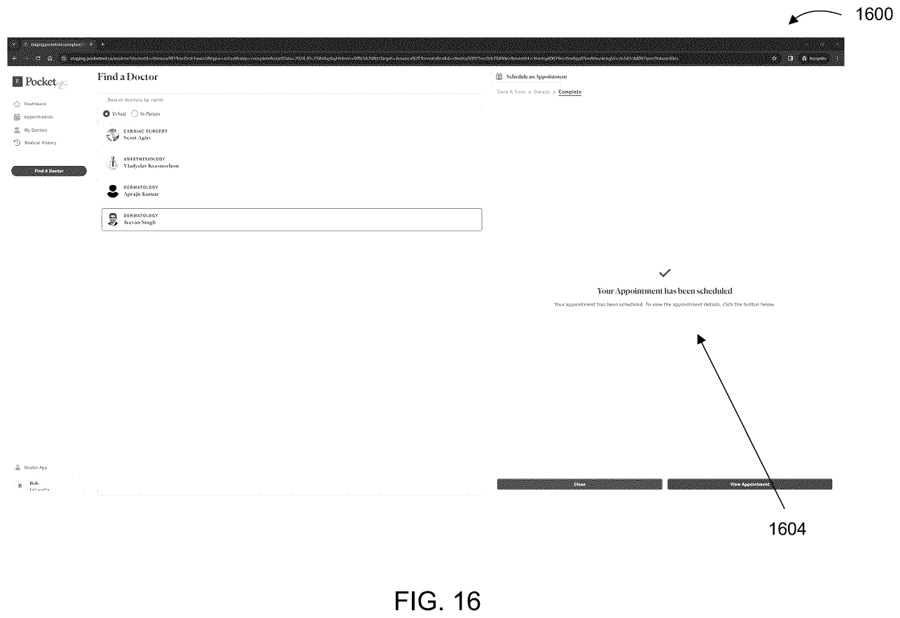

Server 104 may also include input devices that connect to processor 112, such as a keyboard and mouse, as well as output devices, such as a display. Alternatively, or in addition, the input and output devices can be connected to processor 112 via communications interface 108 via another computer device. In other words, input and output devices can be local to server 104 or remote. In the present embodiment, a display may output or provide the example graphical user interfaces in FIGS. 8 to 45. This will be further discussed below.

WAN 128 is an example implementation of the connection between components in system 100, and a person skilled in the art will recognize that WAN 128 is not particularly limited in its configuration. WAN 128 may be any form of network, including a local area network (LAN), or the Internet, and may be accessed by computers, mobile devices or the components of system 100. Computers, such as server 104, patient client terminal 120 and physician client terminal 124 can operate in a networked environment using logical connections to one or more remote computers or other devices, such as a server, a router, a network personal computer, a personal computer, a peer device or other common network node, a wireless telephone or wireless personal digital assistant. In the current embodiment, WAN 128 may be implemented over the Internet. The standards or protocols used for the network may include any form of transmission, such as Transmission Control Protocol/Internet Protocol (TCP/IP), User Datagram Protocol/Internet Protocol (UDP/IP), Hyper Text Markup Language (HTML) and Hyper Text Transfer Protocol (HTTP). In addition, any desired levels and types of security and encryption protocols are contemplated and can be implemented over WAN 128. A person skilled in the art will recognize the different potential network types and different potential network configurations that may be used, along with the different standards and protocols of transmission within the network, and the different forms of security and encryption protocols available. Furthermore, as the data being transmitted between components of system 100, including between server 104, patient client terminal 120, physician client terminal 124 and validation database 132 may be considered sensitive medical data, standards for exchanging healthcare data may be used, including, but not limited to the Fast Healthcare Interoperability Resources (FHIR) standard.

In the current embodiment, as instructed test ordering module 144, prescription module 148, billing module 152 or other instructions to be executed by processor 112, when communicating with either patient client terminal 120, physician terminal 124, validation database 132, or any other servers, databases, computers, or components independent from server 104, processor 112 may communicate using communications interface 108 over WAN 128. As an example, in the current embodiment, prescription module 148 includes physicians providing medications and dosages on a graphical user interface on physician client terminal 124, where the data is then sent via WAN 128 to processor 112 via communications interface 108 to generate a prescription. The generated prescription may then be sent via email or electronic fax via communications interface 108 via WAN 128 to the pharmacy. Other instructions to processor 112 provided by prescription module 148, or other modules, such as test ordering module 144 or billing module 152 may be contemplated, and will also be further explained below. A person skilled in the art will recognize the other potential functions or instructions provided to processor 112 that may use communications interface 108 and WAN 128.

System 100 may include patient client terminal 120 and physician client terminal 124. Patient client terminal 120 and physician client terminal 124 may each be a computer device such as, but not limited to, a desktop computer, a laptop computer, another server, a kiosk, a cell phone, a tablet, a mobile device, a monitor or other suitable device. A person skilled in the art will also appreciate that other, different configurations of patient client terminal 120 and physician client terminal 124 are contemplated. It will also occur to a person skilled in the art that system 100 may include more than one patient client terminal 120 and more than one physician client terminal 124.

Patient client terminal 120 and physician client terminal 124 may include input devices and output devices. In the present embodiment, patient client terminal 120 and physician client terminal 124 may include a display that outputs or provides the example graphical user interfaces in FIGS. 8 to 45.

System 100 may also include validation database 132. Validation database 132 may be any form of database where patient data may be compared to for validation purposes. In the current embodiment, validation database 132 may be a database of insurance information and patient names. As an example, when validating the patient profile data entered into patient database 136, processor 112 may communicate via communications interface 108 over WAN 128 with validation database 132 to compare the patient profile data in patient database 136 with the records of validation database 132. More specifically, the health card number of a patient may be validated against validation database 132 to ensure that patient being entered into patient database 136 does in fact have an insurance plan. As such, any patient who has a validated insurance plan may then have a user account on server 104 to schedule appointments with physicians. In yet another embodiment, validation database 132 may be a credit card validation process for patients who do not have insurance, ensuring that the patient has the ability to pay for medical services despite not having insurance. In other embodiments, validation database 132 may contain physician licensing information to validate whether physicians are in good standing to practice medicine. A person skilled in the art will recognize the different data that validation database 132 may include, and the different potential data from patient database 136 and physician database 140 that may be verified against validation database 132.

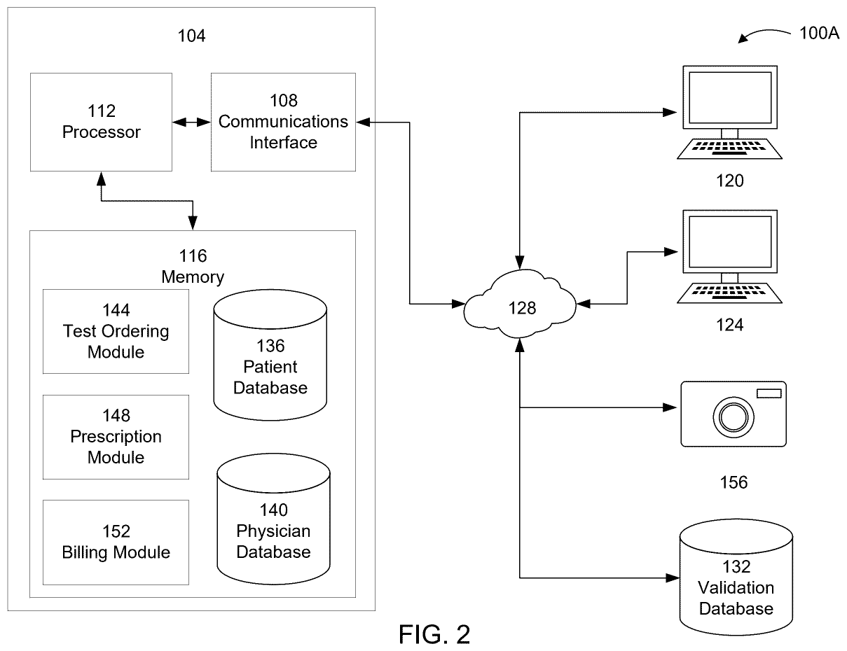

Referring to FIG. 2, system 100A is an alternate embodiment of system 100. The components of system 100A are similar to those of system 100, and are hence similarly numbered. System 100A differs from system 100 in that system 100A further includes camera 156. In the current embodiment, camera 156 may be connected to server 104 via WAN 128. Camera 156 may be used as a sensor to measure a patient's vital statistics, to be either manually or automatically entered into a patient's record in patient database 136. For example, camera 156 may be used to measure the height of the patient.

In other embodiments, camera 156 may be connected to patient client terminal 120, or physician client terminal 124. Scheduled medical appointments between patients and physicians may be conducted electronically over system 100, where the patient uses patient client terminal 120 and the physician uses physician client terminal 124. Camera 156 may be used as a sensor, as mentioned above, or may also be used as a conferencing camera during a virtual medical appointment, allowing the physician to see the patient, along with any physical symptoms to aid in any diagnosis.

Furthermore, in other embodiments, camera 156 may be a different sensor. For example, camera 156 may be a thermal camera to take the temperature of a patient, where the temperature is entered as part of the patient's vitals during a medical appointment to be stored in patent database 136. In yet other embodiments, camera 156 may be other sensors, such as an oxygen saturation sensor to measure the percentage of oxygen saturation in a patient's blood. Other sensors contemplated may include blood pressure sensors, weight sensors, respiratory sensors and hear rate sensors. Sensors are not limited to being used in virtual appointments, but may also be connected to the physician client terminal 124 to be used as part of an in-person medical appointment. It will occur to a person skilled in the art that any combination of any of the aforementioned sensors may be part of system 100. A person skilled in the art will also recognize the other potential sensors to measure the vitals of a patient that may be part of system 100. Furthermore, a person skilled in the art will recognize the different combinations and layouts for connecting any number of sensors and camera 156 to each of server 104, physician client terminal 124, patient client terminal 120 and WAN 128 either separately or in combination with each other.

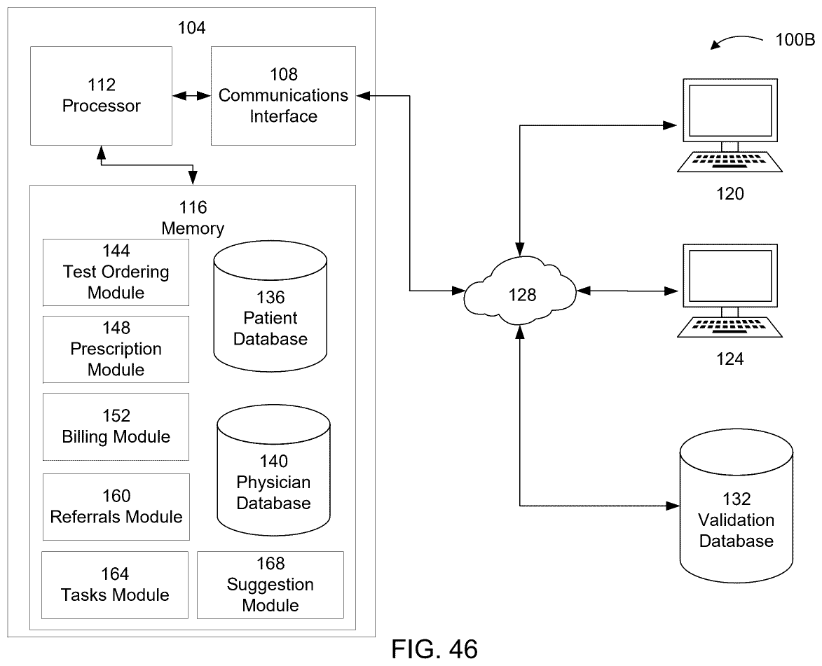

Referring to FIG. 46, system 100B is yet another embodiment of system 100. The components of system 100B are similar to those of system 100, and are hence similarly numbered. System 100B differs from system 100 in that memory 116 of system 100B further includes referrals module 160, tasks module 164, and suggestion module 168. Referrals module 160 provides instructions to processor 112 to generate referral requests for patients to see other medical professionals. Tasks module 164 provides a checklist or a list of tasks for a physician or medical professional to perform during a scheduled medical appointment. Suggestion module 168 provides suggestions or alerts to the physician based on the medical data of the patient. The referrals module 160, tasks module 164 and suggestion module 168 will be further discussed below.

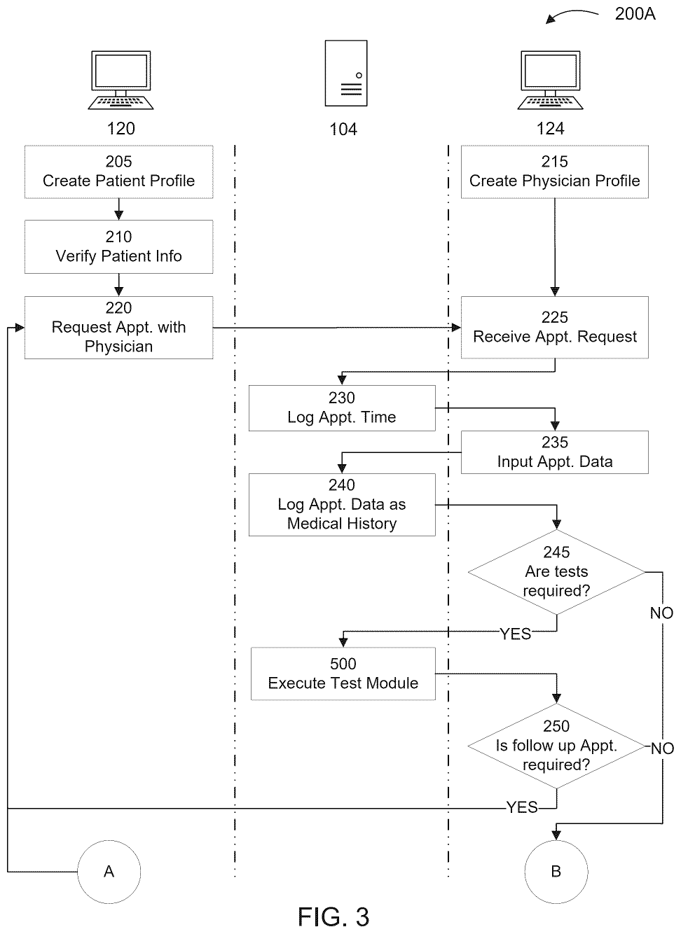

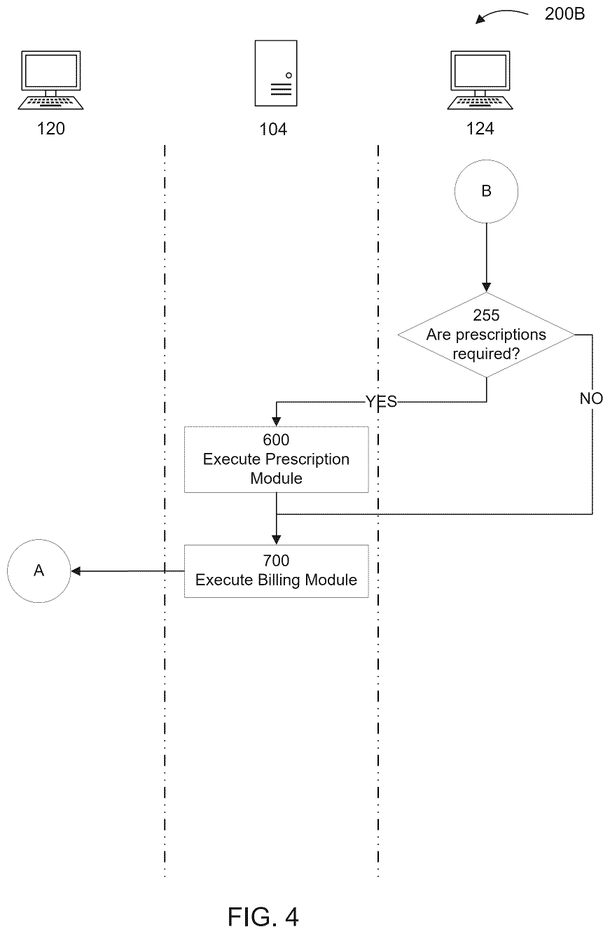

Referring to FIGS. 3 and 4, an example method 200A and 200B of using system 100 is provided. Specifically, methods 200A and 200B depict the scheduling and performance of a medical appointment, and also the storing of details related to said medical appointment in patent database 136 as an electronic medical record. As is depicted in FIGS. 3 and 4, the example method of 200A and 200B include steps that are performed by patient client terminal 120, server 104 and physician client terminal 124.

Block 205 depicts the creation of a patient profile on patient client zero. In the current embodiment, the patient is presented with a graphical user interface to create a new patient profile to which their patient information will be associated with. Specifically, the patient profile may be saved as a record in patient database 136.

Referring to FIG. 8, screenshot 800 depicts an example graphical user interface for a patient to enter or edit the patient's personal information 804, also referred to herein as a patient's biographic data 804, as part of the patient's settings or patient's profile. As is depicted in screenshot 800, creation of a patient profile may include entering a patient's email address, along with the patient's personal information 804, including, but not limited to a first name, middle name, last name, date of birth and sex.

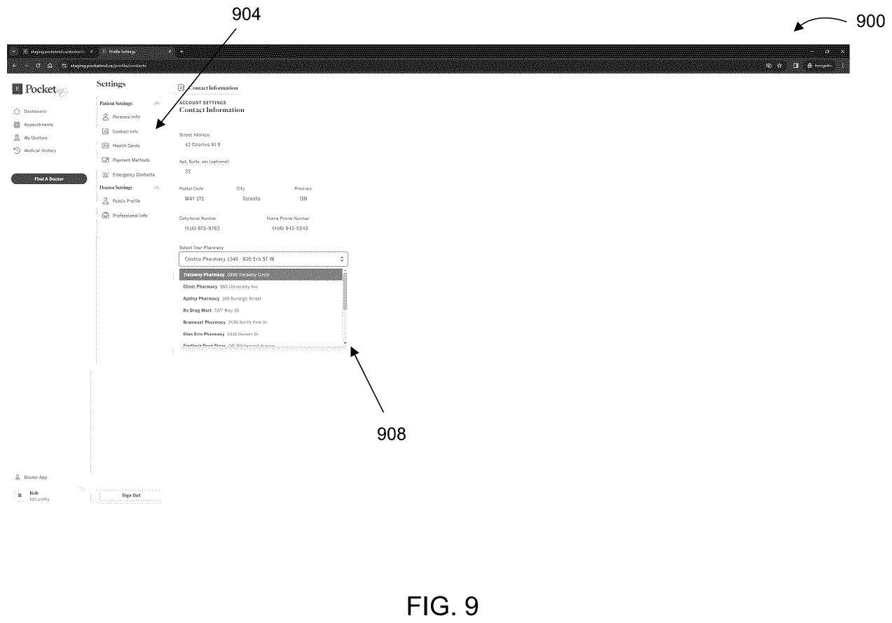

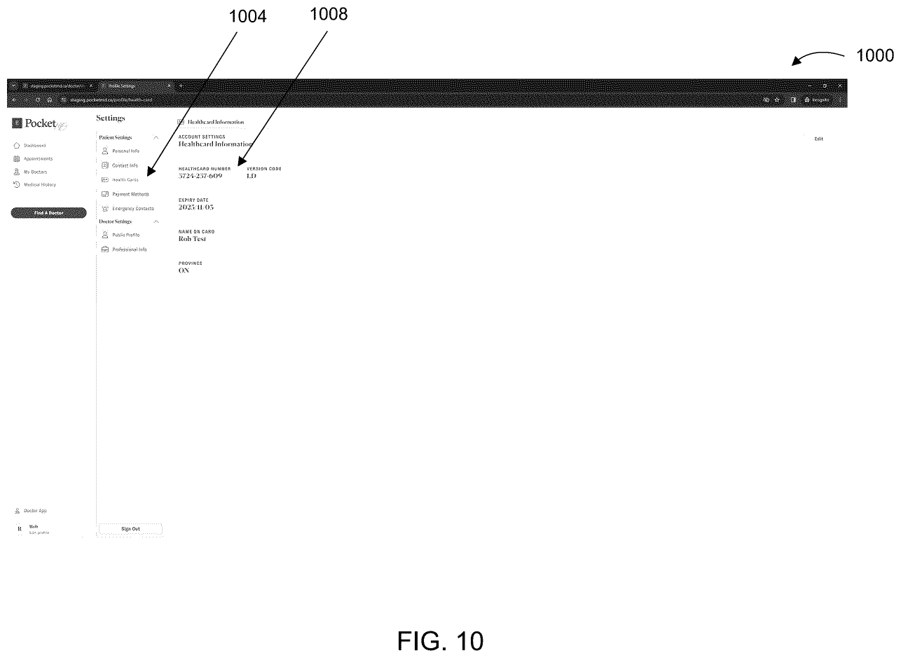

Referring to FIG. 9, screenshot 900 depicts further example information that may be entered into an example graphical user interface for the creation or edit of a patient's profile. Specifically, a patient's contact information 904 may be part of the creation of a patient's profile and may include the patient providing a patient address and contact information. Furthermore, a patient may also provide the pharmacy of their choice at pharmacy selection 908. A patient may select more than one pharmacy under pharmacy selection 908, allowing the patient to pickup their medication at a pharmacy of their choice based on their location. As an example, a patient's home may be located near a first pharmacy, but a patient's workplace may be located near a second pharmacy. As such, allowing a patient to select more than one pharmacy provides better flexibility for the patient to pick up their medication based on their schedule. Alternatively, if a patient decides to relocate or move their primary residence, the patient may select new pharmacies based on their new residence location.

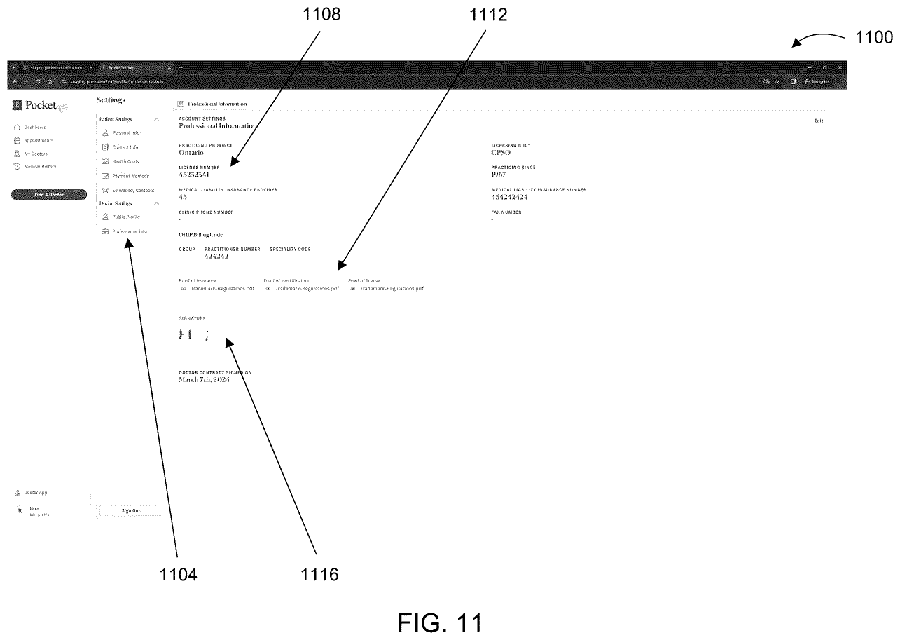

Referring to FIG. 10, screenshot 1000 depicts further example information that may be entered into an example graphical user interface for the creation or edit of a patient's profile. Specifically, a health card or insurance information 1004 may be provided towards the creation of a patient's profile. In the current embodiment, the health card numberer 1008 may be entered and may be compared against validation database 132, so as to ensure that the patient associated with the patient profile does indeed have medical insurance. A person skilled in the art will also recognize that other information associated with the patient profile, such at those provided in screenshots 800, 900 and 1000 in FIGS. 8 to 10 may all be used to validate a patient, to ensure that the patient is indeed a real person creating the patient profile, and not a fictitious user account or a bot. Additionally, verifying patient information against validation database 132 also provides a verification that the patient can pay for the medical services. Specifically, insurance information may be validated, and if the patient does not have medical insurance, then a payment method, such as a credit card, may be validated. The verification of patient info is depicted at block 210 of FIG. 3. In some embodiments, a patient profile may not be able to be created without the verification being successful. In yet other embodiments, patients may be prevented from scheduling a medical appointment without the verification being successful.

Screenshots 800 to 1000 of FIGS. 8 to 10 also provide a graphical user interface to add payment methods for paying medical bills, and the bibliographic information of any emergency contacts, and the relationship of said emergency contact to the patient. A person skilled in the art will recognize the different information that may be provided in association with a patient toward the creation and/or edit of a patient profile to be stored in patient database 136.

Referring to FIG. 3, block 215 depicts the creation of a physician profile, where the data associated with a physician is stored on physician database 140. Referring to FIG. 11, screenshot 1100 depicts an example graphical user interface for a physician to enter or edit the physician's professional information 1104. In the current embodiment, a physician's professional information 1104 may include, but is not limited to the practicing province, the license number 1108 of the physician, documentation 1112 providing proof of insurance, proof of identification and proof of license, and the signature 1116 of the physician. Other information associated with the physician may also be contemplated to be entered into the graphical user interface as part of the physician profile.

While not depicted in the current example method 200A and 200B of FIG. 3 and FIG. 4, any of the physician professional information 1104 may also be validated against validation server 132. For example, the license number 1108 of the physician may be validated to ensure that the practicing physician is in good standing. A person skilled in the art will recognize the different physician professional information 1104, or any information that may be associated with the physician stored in physician database 140 may be validated against validation server 132.

Documentation 1112 may be uploaded as PDFs or any other file format to be stored associated with the record of the physician in physician database 140. Physician signature 1116 may be used for attaching to test requests and/or prescriptions.

Referring to FIG. 2, block 220 depicts the patient client terminal 120 requesting an appointment with the physician. A patient may schedule an appointment with any physician using system 100 by selecting the physician through the graphical user interface and scheduling and appointment based on the availability of the physician and the physician's calendar.

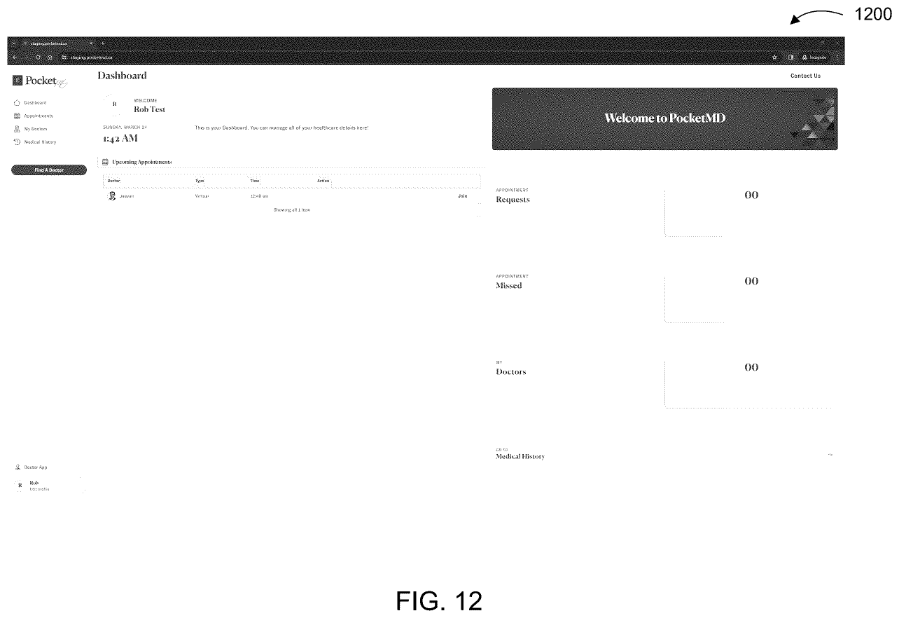

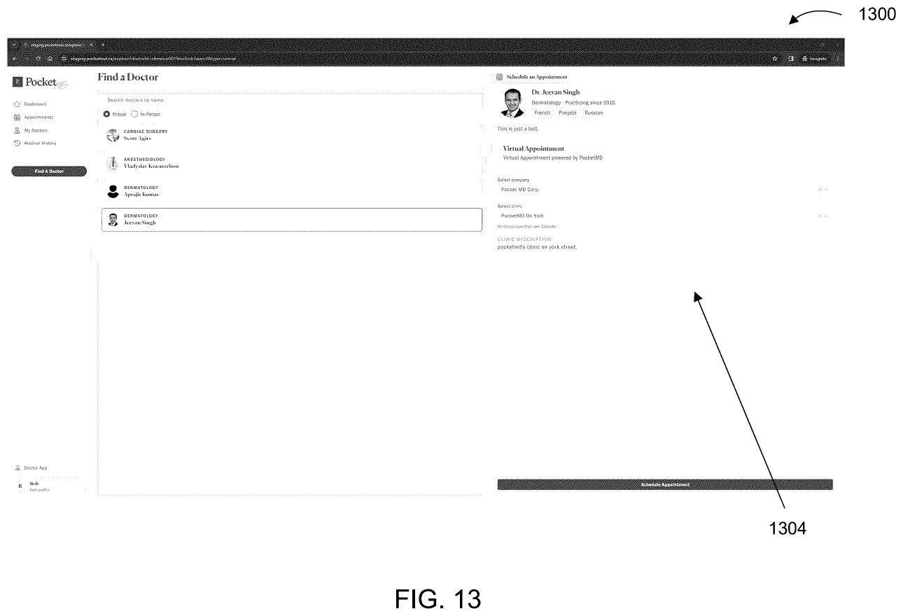

Referring to FIG. 12, in the current embodiment, screenshot 1200 is a graphical user interface of a dashboard for the patient on patient client terminal 120. The dashboard may indicate the upcoming appointments, the appointment requests, missed appointments and the number of physicians that the patient has seen. Referring to FIG. 13, in the current embodiment, screenshot 1300 is a graphical user interface of scheduling an appointment after a physician has been selected. In tab 1304, the company and clinic of the physician may be selected. A physician may work at more than one company, and/or with more than one clinic. To that end, a patient may select which company or clinic they wish to attend for the virtual or in person appointment.

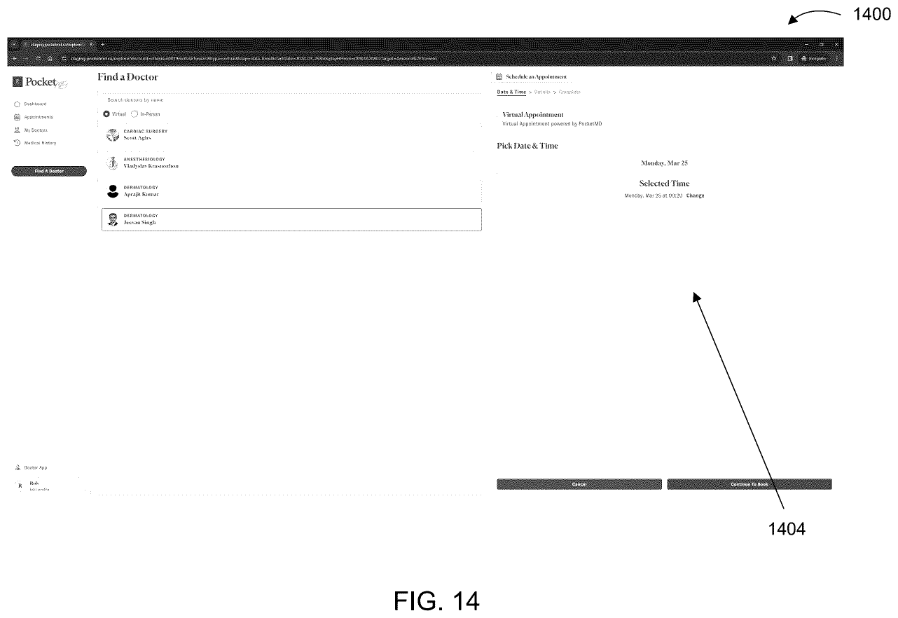

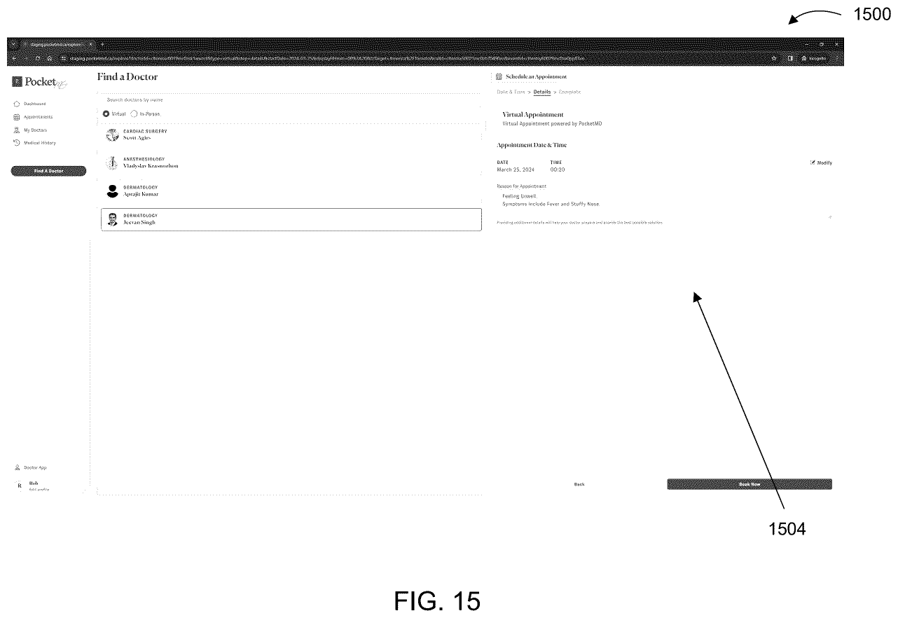

Referring to FIG. 14, screenshot 1400 is an example graphical user interface where a patient selects the date and time at tab 1404 for scheduling the virtual or in person appointment. Referring to FIG. 15, screenshot 1500 is an example graphical user interface where the patient may provide the reasoning for requesting the medical appointment. A person skilled in the art will recognize that other information may be requested from the patient when scheduling an appointment. Once all information has been collected and submitted, tab 1604 of screenshot 1600 of FIG. 160 provides confirmation that the appointment has been scheduled. The confirmation indicates that server 104 and physician client terminal 124 have received the appointment request from patient client terminal 120, as is depicted at block 225 of FIG. 3.

In alternate embodiments, as opposed to the patient using patient client terminal 120 at block 220 to request an appointment with the physician, and the physician client terminal 124 receiving the appointment request at block 225, the physician may be the originator of the request for an appointment. Specifically, the physician may request a medical appointment using physician client terminal 124 with the patient, and the patient client terminal 120 will receive the request for the medical appointment. A physician may request a medical appointment due to various reasons including, but not limited to, following up with a patient after receiving test results, following up with a patient after the patient has seen another medical professional, or based on an alert provided by suggestion module 168. This will be further discussed below.

Referring to FIG. 3, block 230 depicts sever 104 logging the appointment time. The appointment time is logged and associated with the patient in patient database 136. In alternate embodiments, appointment time may be logged in a log database (not shown), as a history of events associated with either patients or physicians.

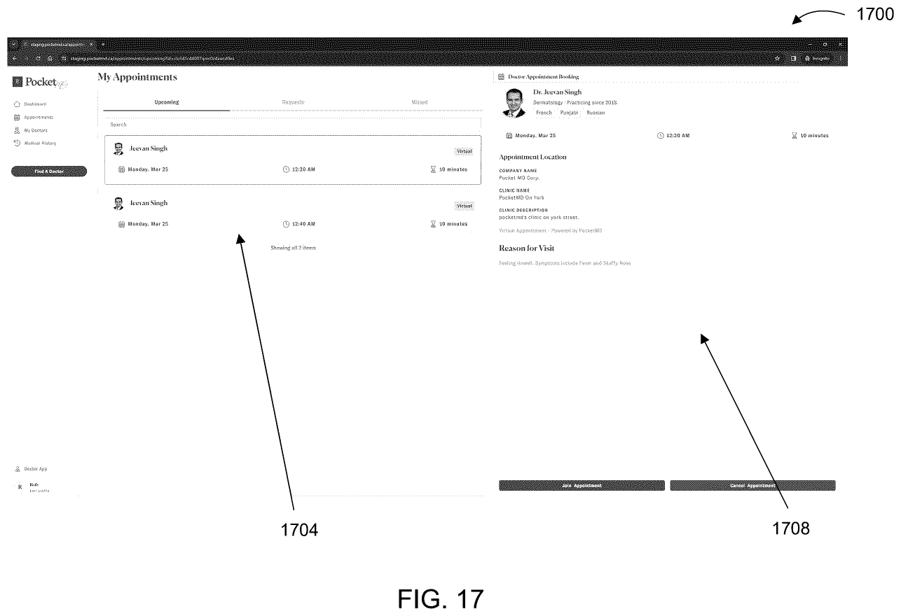

Referring to FIG. 17, screenshot 1700 is an example graphical user interface where the appointments and/or the log of appointments may be accessed on server 104 from patient client terminal 120. As can be seen, tab 1704 provides a listing of upcoming appointments, and may be adjusted to showcase requested appointments or missed appointments. Tab 1708 provides details of the specific appointment. In alternate embodiments, tab 1704 or elsewhere in the graphical user interface, a history or log of the appointments may also be displayed.

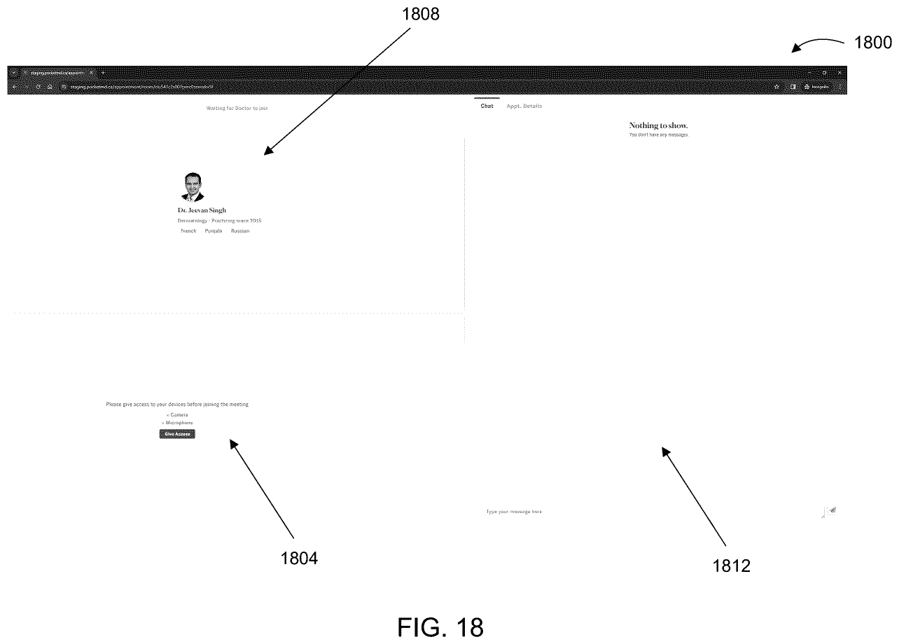

When the scheduled appointment time arrives, the patient can attend the appointment either virtually or in person depending on the appointment type when creating/scheduling the appointment. In the event that a virtual appointment occurs, a patient can access the graphical user interface depicted at screenshot 1800 on FIG. 18, and commence the virtual appointment. At tab 1808, the physician's camera will show the physician, and at tab 1804, the patient's camera will show the patient. Tab 1812 provides an area to chat between the physician and patient, however similar to known web conferences, audio communication is also available through microphones and audio devices on the patient client terminal 120 and the physician client terminal 124.

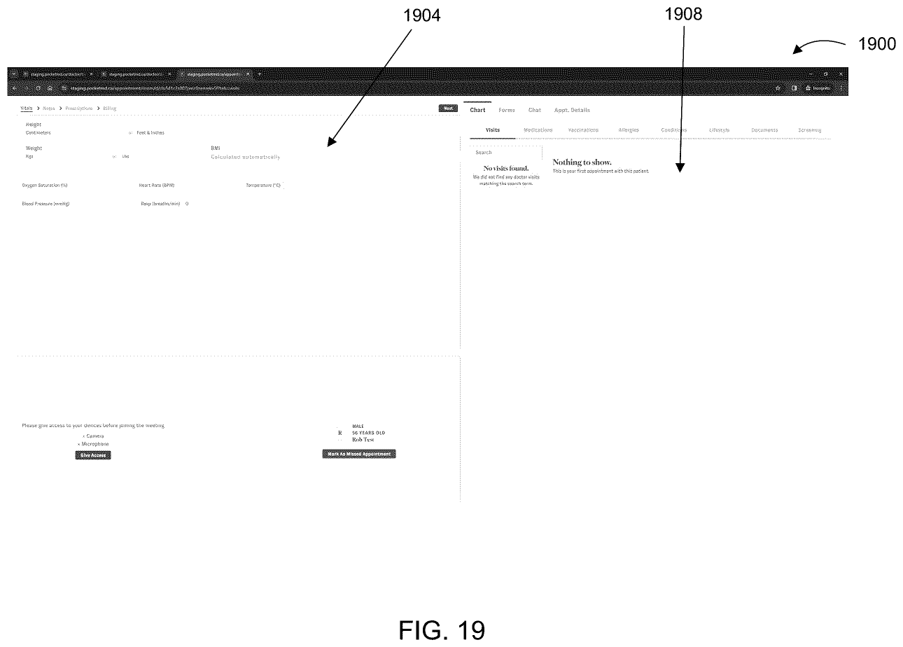

Referring to FIG. 19, screenshot 1900 provides a graphical user interface from the physician client terminal 124 where the physician is able to access various tabs to input and provide appointment data. Appointment data is data associated with the appointment with the patient, and may include vitals, and notes. Vitals may be input manually into the vitals tab 1904 by the physician, or may be provided automatically through camera 156 or alternate sensors. As mentioned previously, with a virtual appointment, system 100A may use camera 156 to measure the height of the patient, or other sensors to record and provide the vitals to server 104 to be recorded automatically into fields on vitals tab 1904. Alternatively, if it is an in person appointment, camera 156 or other sensors may record the vitals and send the data to server 104 via physician client terminal 124 and automatically populate fields in vitals tab 1904. A person skilled in the art will recognize that other vitals may be contemplated, and different sensors may also be contemplated for recording said vitals. Appointment data may also include notes, which can also be added during the appointment on the graphical user interface. Referring to FIG. 3, block 235 depicts inputting said appointment data.

Block 240 depicts logging appointment data as medical history associated with the patient in patient database 136. As such, patients can access their medical history and appointment data for each appointment they attend with a physician anytime after the appointment using patient client terminal 120. Similarly, physicians can access the medical history and appointment data for a patient from patient database 136 using physician client terminal 124.

In addition to vitals, other historical data or appointment data may be added to patient database 136 to be associated with the patient. Referring to FIG. 19, tab 1908 allows for physician to navigate between different tabs, including charts, forms the chat tab 1812, and the appointment details. Furthermore, in the current embodiment, within the chart tab, the physician can navigate between both historical data and appointment data. Specifically, the physician can navigate between data pertaining to previous visits, medications, vaccinations, allergies, conditions, lifestyle, documents and screening. More specifically, the physician can use the graphical user interface and navigate between the aforementioned tabs to review historical data for the patient, and add new data pertaining to the latest appointment, hence updating the patient's medical file. Alternatively, if the patient has visited previous clinics and physicians, but does not have an electronic medical file, or a historical record on patient database 136, the physician can add details pertaining to the patient's medical history. In its current configuration in FIG. 19, tab 1908 provides a list of previous visits that the patient may have had to various clinics or other physicians.

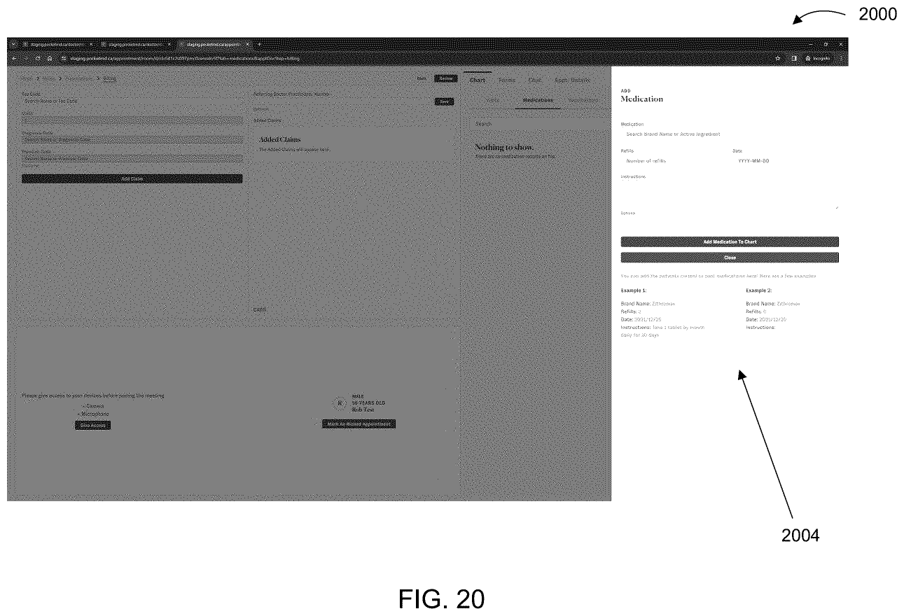

Referring to FIG. 20, screenshot 2000 depicts an example of adding a new medication record associated with the patient to patient database 136 at tab 2004. As can be seen, in the current embodiment, this includes adding the name of the medication, the number of refills, the date the medication was prescribed, and the instructions for taking the medication. A person skilled in the art will recognize that medication records are not limited to the above-mentioned fields, and that other fields may be contemplated for providing details regarding medication taken or to be taken by the patient.

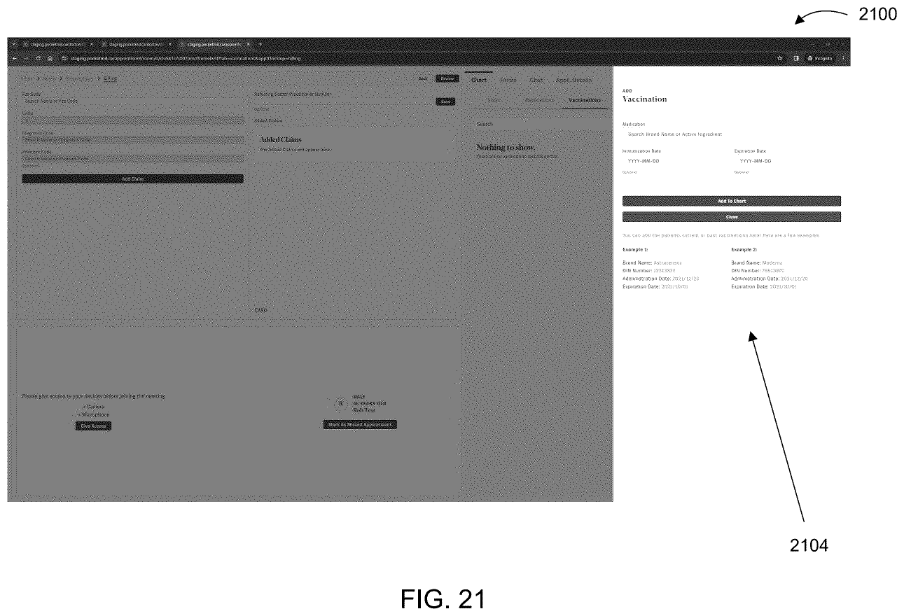

Referring to FIG. 21, screenshot 2100 depicts an example of adding a new vaccination record associated with the patient to patient database 136 at tab 2104. As can be seen in the current embodiment, this includes adding the medication, immunization date and the expiration date. A person skilled in the art will recognize that vaccination records are not limited to the above-mentioned fields, and that other fields may be contemplated for providing details regarding vaccinations taken or to be taken by the patient.

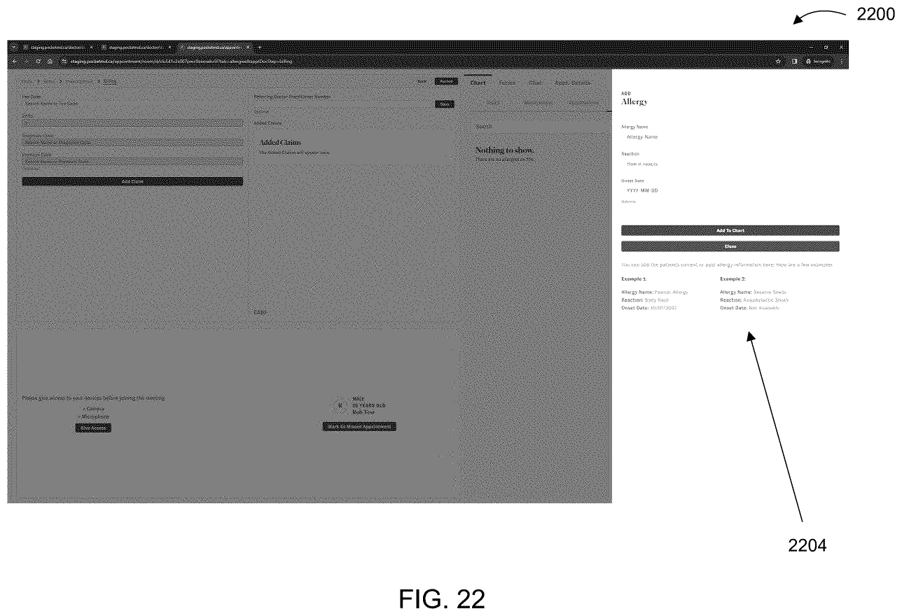

Referring to FIG. 22, screenshot 2200 depicts an example of adding a new allergy record associated with the patient to patient database 136 at tab 2204. As can be seen, in the current embodiment, this includes adding the allergy name, the allergy reaction, and the onset date of the allergy. A person skilled in the art will recognize that allergy records are not limited to the above-mentioned fields and that other fields may be contemplated for providing details regarding allergies discovered or previously had by the patient.

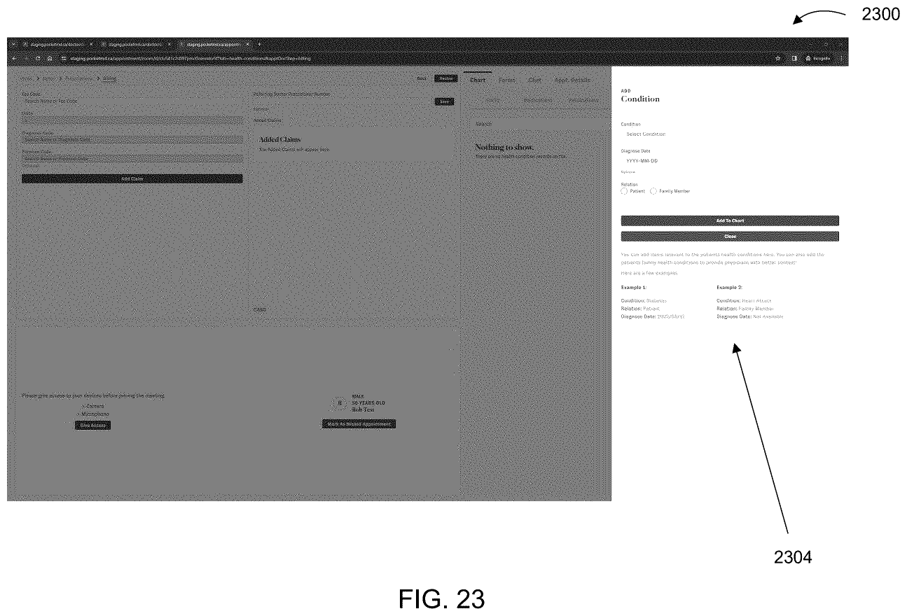

Referring to FIG. 23, screenshot 2300 depicts an example of adding a new condition record associated with the patient to patient database 136 at tab 2304. As can be seen, in the current embodiment, this includes adding the condition, name, the diagnosis state, and whether the condition is related to the patient or a family member. A person skilled in the art will recognize that any condition records are not limited to the above-mentioned fields and the other fields may be contemplated for providing details regarding conditions discovered or previously discovered by the patient or the patient's family members.

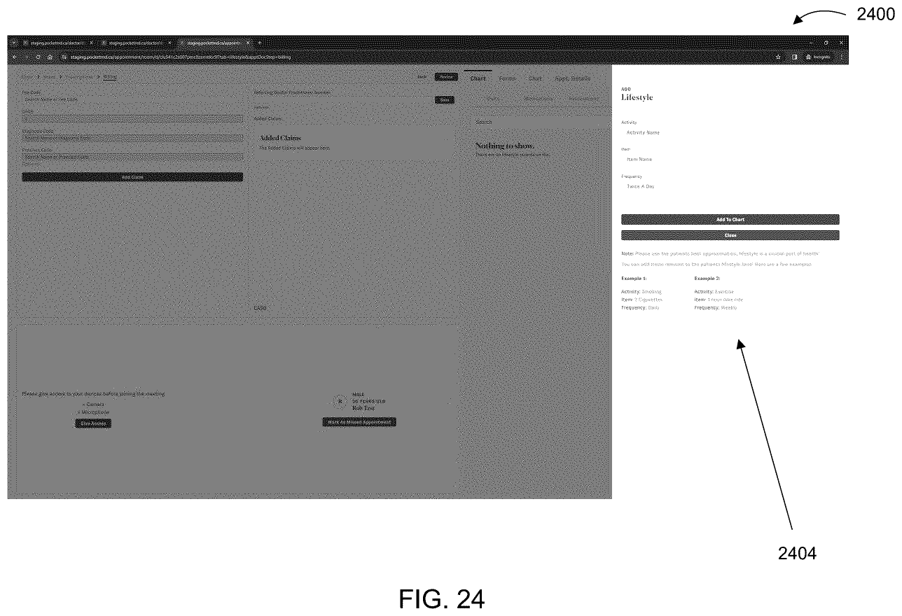

Referring to FIG. 24, screenshot 2400 depicts an example of adding a new lifestyle record associated with the patient to patient database 136 at tab 2404. As can be seen, in the current embodiment, this includes adding the lifestyle activity, and associated item with the activity, and a frequency that the lifestyle activity occurs. A person skilled in the art will recognize that any lifestyle record is not limited to the above-mentioned fields and that other fields may be contemplated for providing details regarding lifestyles previously undertaken by the patient or new lifestyle activities that the patient recently undertook.

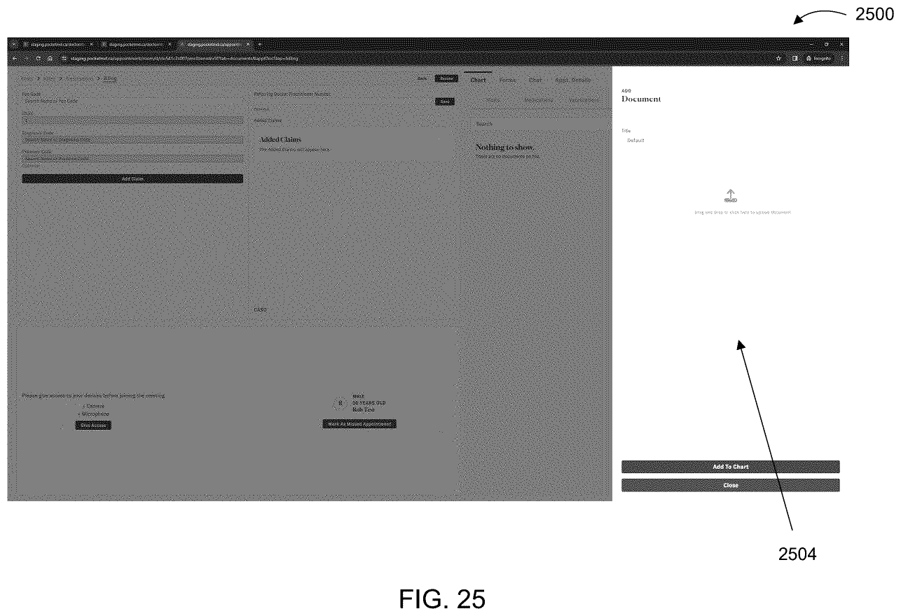

Referring to FIG. 25, screenshot 2500 depicts an example of adding a relevant document associated with the patient to patient database 136 at tab 2504. As can be seen, in the current embodiment, this includes dragging and dropping, and/or uploading the document to tab 2504. A person skilled in the art will recognize that any files uploaded associated with the patient record in patient database 136 are not limited to document file types but may be a file of any file type. For example, pictures or videos may also be uploaded.

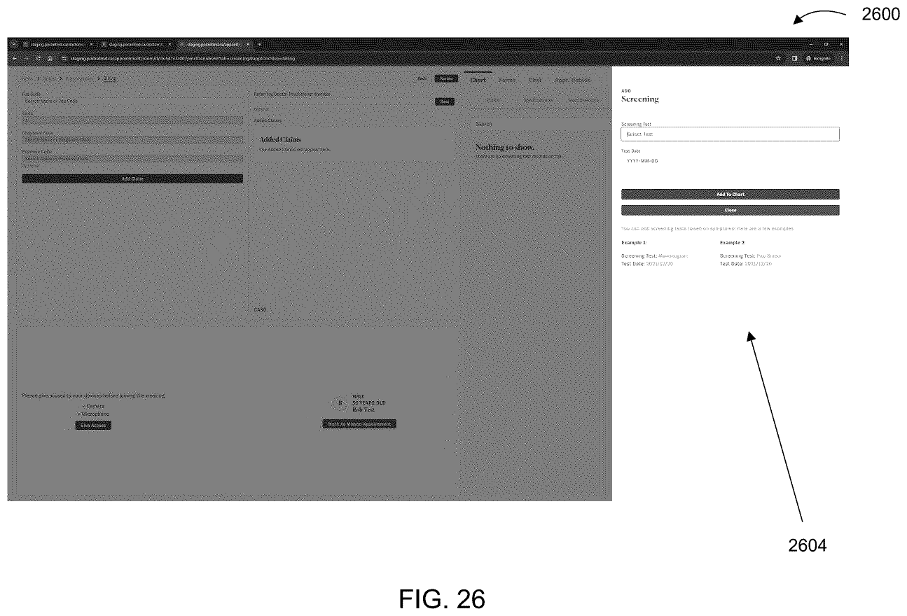

Referring to FIG. 26, screenshot 2600 depicts an example of adding a screening test associated with the patient to patient database 136 at tab 2604. As can be seen, in the current and embodiment, this includes adding a screen test type and a test date. A person skilled in the art will recognize that the screen test record is not limited to the above-mentioned field and that other fields may be contemplated for providing details regarding screen tests that the patient previously undertook or plans to undertake.

Referring to FIG. 3, block 245 depicts a determination by the physician as to whether medical tests are required to help diagnose or treat the patient. If the physician deems that a medical test is required, processor 112 may execute instructions provided from test ordering module 144. If the physician deems that a medical test is not required, the next step is at block 255 to determine whether a prescription is needed.

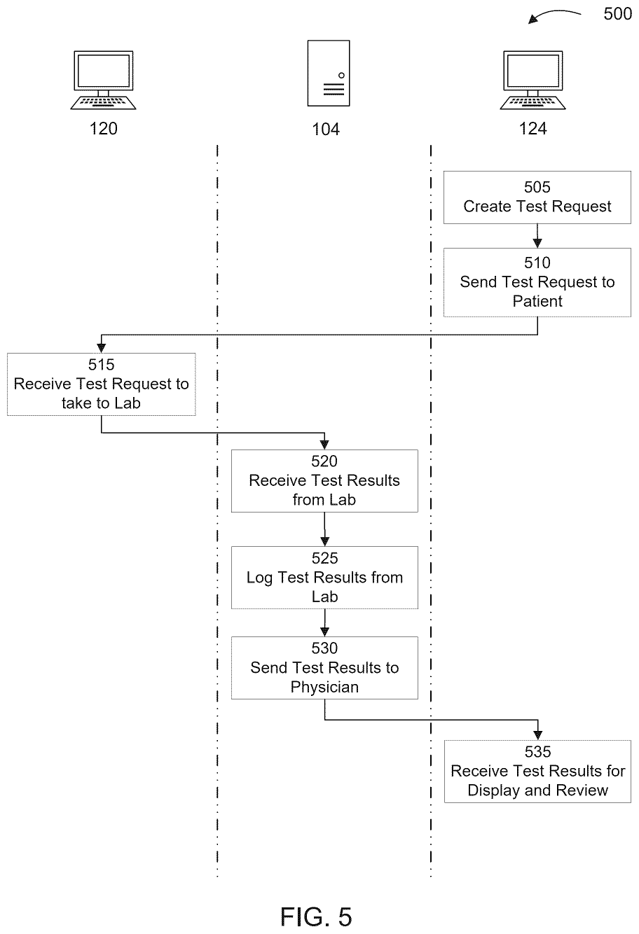

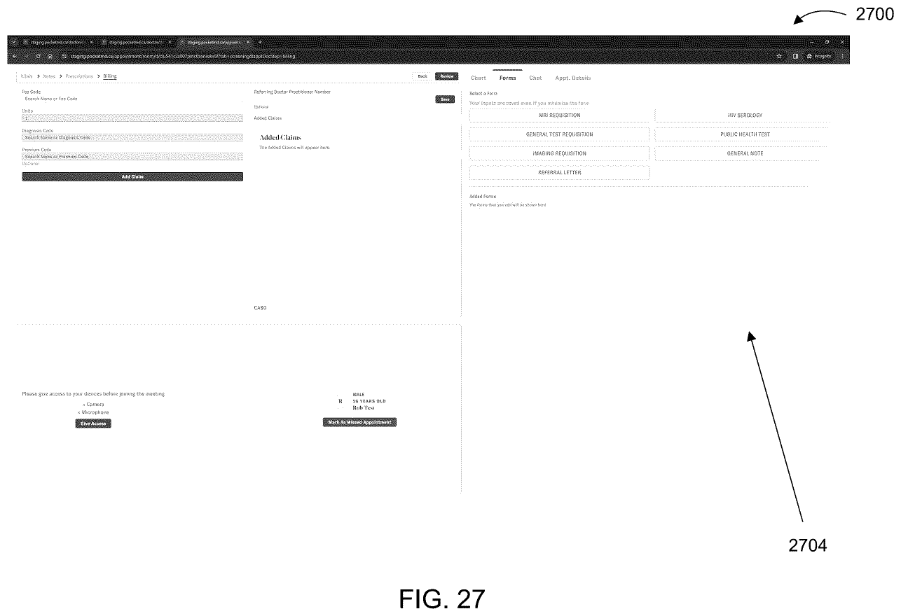

Referring to FIG. 5, block 500 may include the below described substeps. Specifically, block 500 may include block 505, where the test request is created. Potential tests that may be ordered include, but is not limited to, a MRI requisition, a HIV serology, a general test requisition, a public health test, an imaging requisition, a general note and a referral letter. Referring to FIG. 27, screenshot 2700 depicts the forms tab, which provides the above-mentioned options at tab 2704. A person skilled in the art will recognize that other medical tests or procedures may be contemplated, and the graphical user interface may be expanded to allow for said other medical tests or procedures.

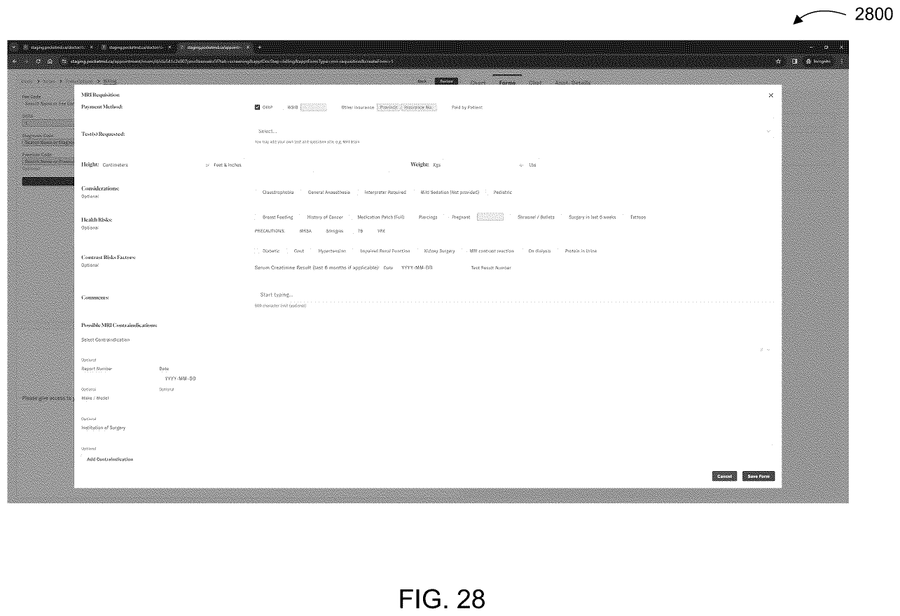

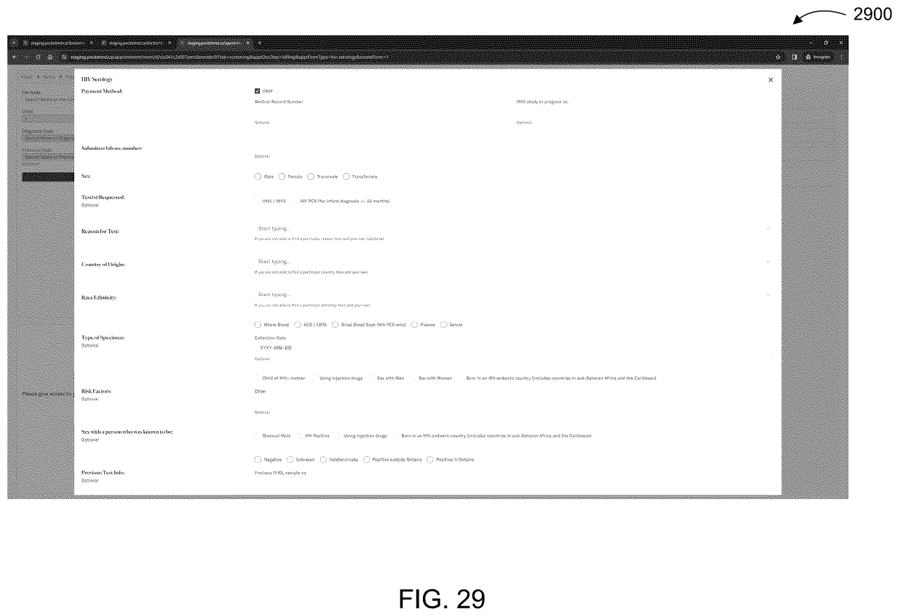

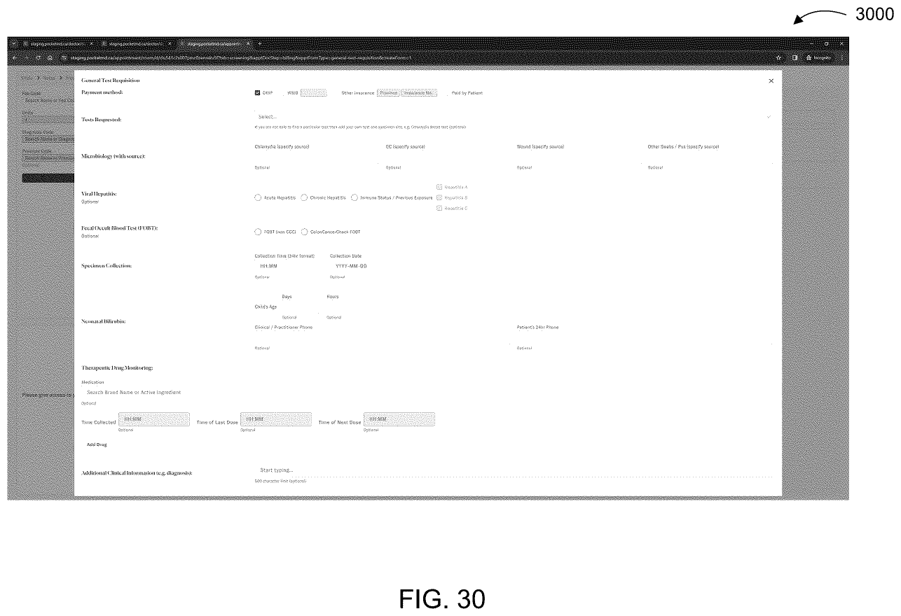

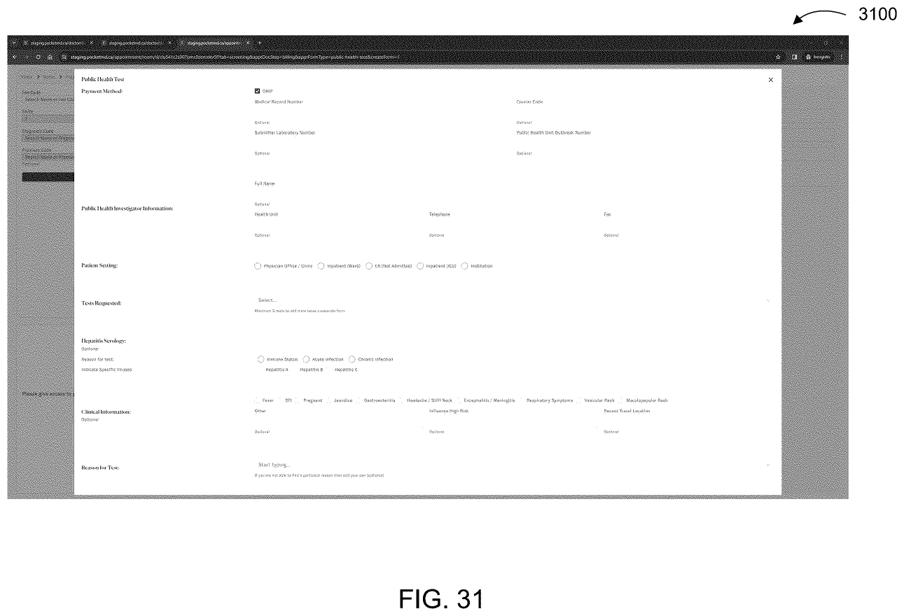

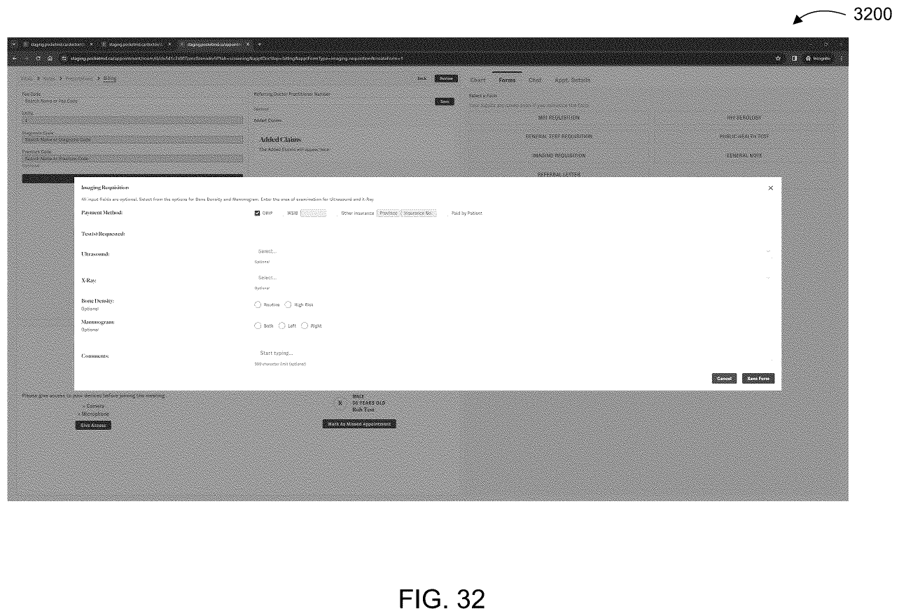

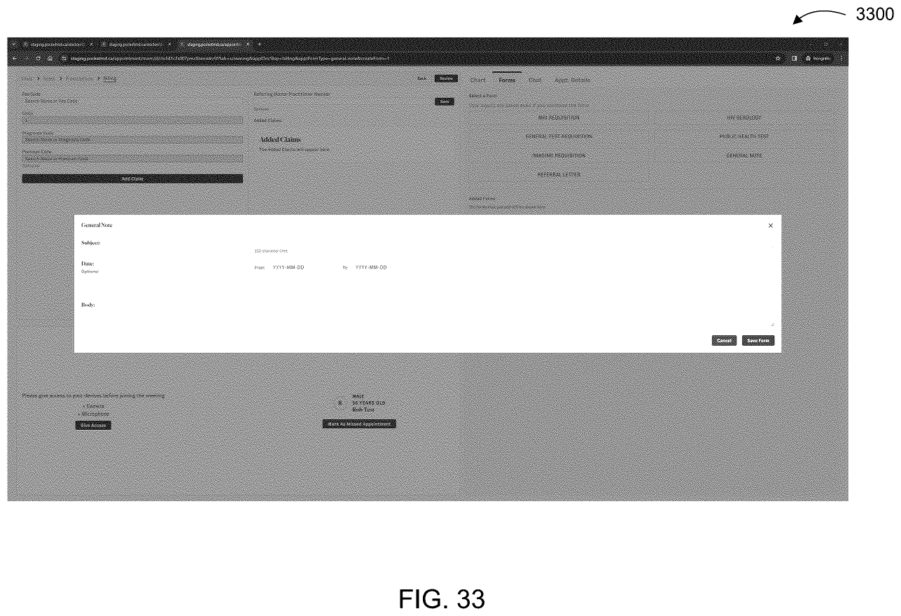

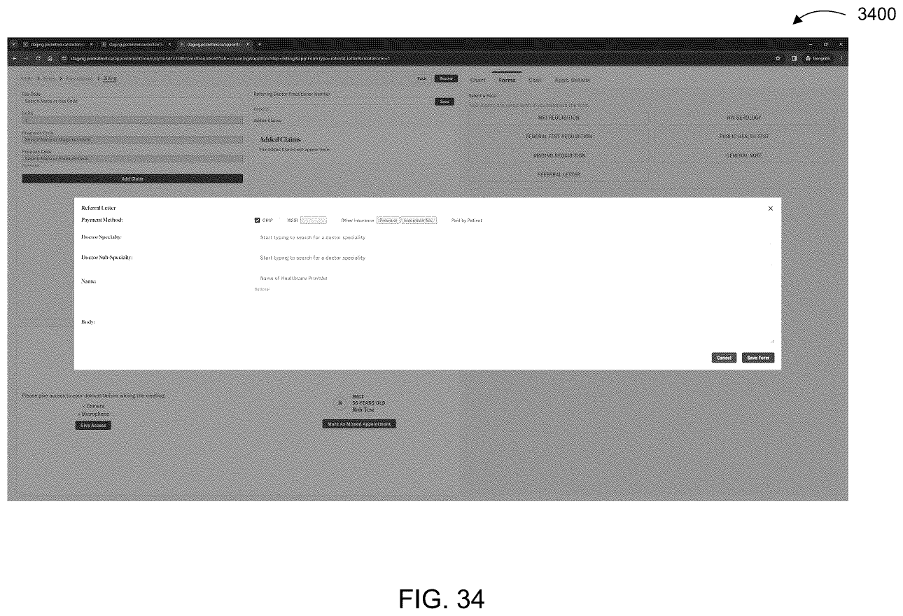

Referring to FIGS. 28 to 34, example graphical user interfaces are depicted for inputting information to request a medical test or procedure. Specifically, FIG. 28 includes screenshot 2800 which depicts an example graphical user interface or electronic form for requesting an MRI for a patient. Referring to FIG. 29, screenshot 2900 depicts an example graphical user interface or electronic form for requesting an HIV serology. Referring to FIG. 30, screenshot 3000 depicts an example graphical user interface or electronic form for a general test requisition. Referring to FIG. 31, screenshot 3100 depicts an example graphical user interface or electronic form for requesting a public health test. Referring to FIG. 32, screenshot 3200 depicts an example graphical user interface or electronic form for an imaging requisition. Referring to FIG. 33, screenshot 3300 depicts an example graphical user interface or electronic form for including a general note for providing instructions related to requesting medical tests that may not be listed in the example graphical user interface. Referring to FIG. 34, screenshot 3400 depicts an example graphical user interface or electronic form for providing a referral letter for the patient to see another physician, specialist, or any other medical professional.

A person skilled in the art will recognize that the fields provided in any of the example graphical user interfaces or electronic forms in FIGS. 28 to 34 are not limited to the variables, labels, or text fields provided in screenshots 2800, 2900, 3000, 3100, 3200, 3300 and 3400. Rather any number of fields or variables in relation to the medical test or procedure requested may be contemplated and be placed on the graphical user interface as a prompt to request information from the physician using the graphical user interface or filling in the electronic form on physician client terminal 124 or on server 104.

Upon completion of filling in the respective test request form, the test request form is sent to the patient to be displayed and/or accessed from patient client terminal 120. This is depicted at block 510 of FIG. 5. At block 515, the test request form is received by the patient client terminal 120. The patient may print a hardcopy of the completed test request form to take to the lab or relevant medical professional. In alternate embodiments, the completed test request form may be electronically sent or faxed to the lab or relevant medical professional. Furthermore, in alternate embodiments, the completed test request form may be sent directly to the lab or relevant medical professional by server 104. A person skilled in the art will recognize the different potential methods of scheduling medical tests with labs or other medical professionals and how the request for the test from the physician is provided to said labs or other medical professionals.

At block 520, after the requested medical test or procedure has been completed, the test results may be received electronically by server 104. Server 104 may receive the test results via fax, the test results may be uploaded onto server 104, or alternatively, an application programming interface (API) may be used to either retrieve the test results to be provided to server 104, or the test results may be provided to server 104 through an API. In alternative embodiments, server 104 may receive the test results manually, where the medical test or procedure results are either sent to the patient or the physician, upon which the data may be entered into server 104 via either patient client terminal 120 by the patient or physician client terminal 124 by the physician or the physician's staff.

At block 525, the test results are logged as part of the medical history of the associated patient, and the test results are saved as part of the patient's record in patient database 136. In the current embodiment, as the test results are received directly by server 104, at block 530, the test results are sent to the physician for display on physician client terminal 124 for the physician's review at block 535. However, in alternate embodiments, where the test results are received by the physician and input into server 104 via physician client terminal 124, the physician client terminal 124 may still access and display the test results for review by the physician.

Referring to FIG. 2, block 250 depicts the step of the physician determining whether a follow up medical appointment is necessary after reviewing the test results. If the physician deems that a follow up medical appointment is warranted, the patient may be sent a message from the physician, and the patient may schedule another appointment at block 220. If a follow up medical appointment is not required, then at block 255 the physician may determine whether a prescription is required for the patient.

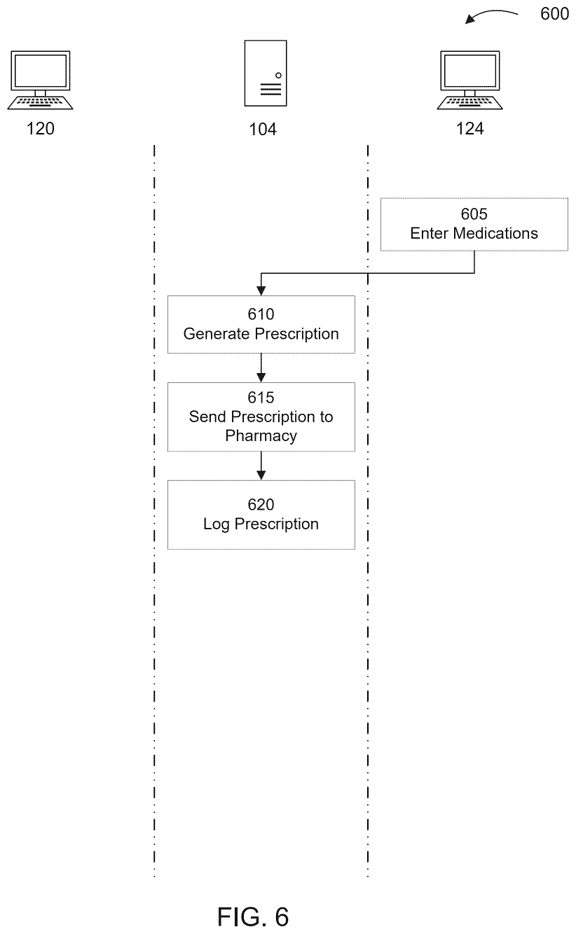

If a prescription is needed, processor 112 may execute the instructions provided by prescription module 148 at block 600. Referring to FIG. 6, the instructions provided by prescription module 148 may include block 605, where a physician may provide a prescription for the medications that the patient may require.

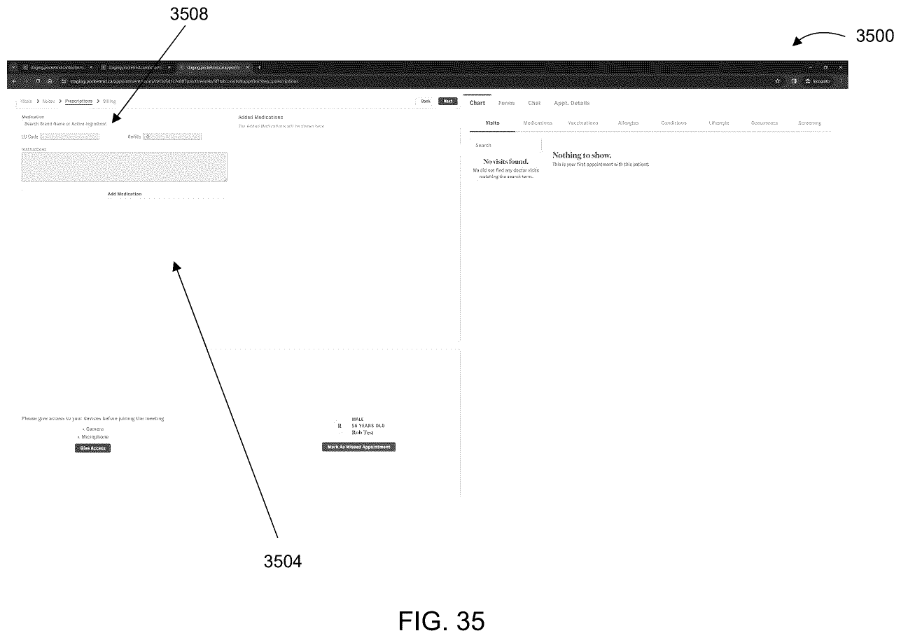

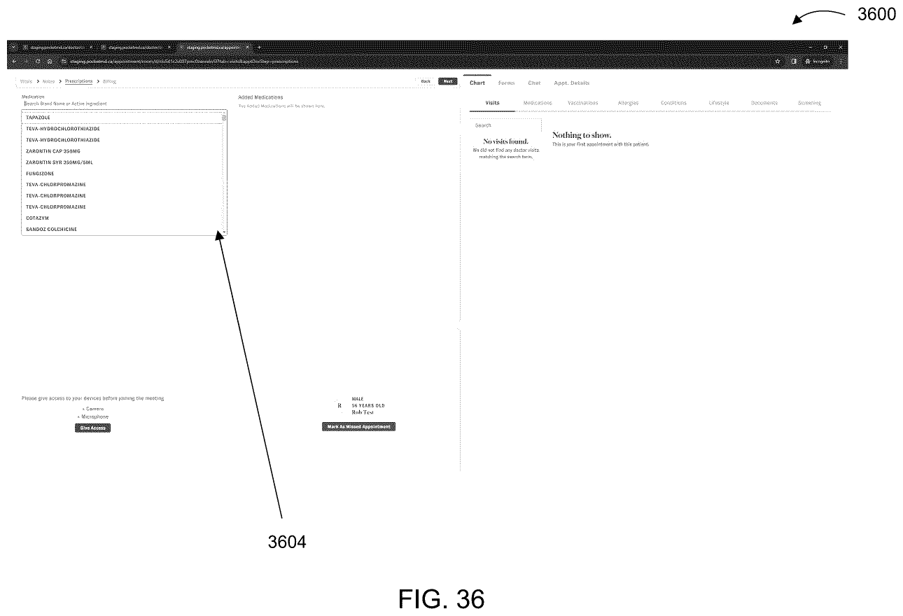

Referring to FIG. 35, screenshot 3500 is an example graphical user interface on physician client terminal 124 or server 104 for the physician to enter medications that are to be prescribed to the patient. Specifically tab 3504 provides various fields for the physician to fill out with respect to the medication that is to be prescribed. In the current embodiment, field 3508 provides the physician with a searchable drop down menu of medications that the physician may prescribe. Referring to FIG. 36, screenshot 3500 shows this drop down menu as field 3508. A person skilled in the art will recognize that different fields are contemplated for the creation of prescriptions. For example, the creation of prescriptions may include a field requiring the physician to provide an electronic signature for every prescription created, so as mimic the signing of a hardcopy prescription on a prescription pad. Other fields are contemplated.

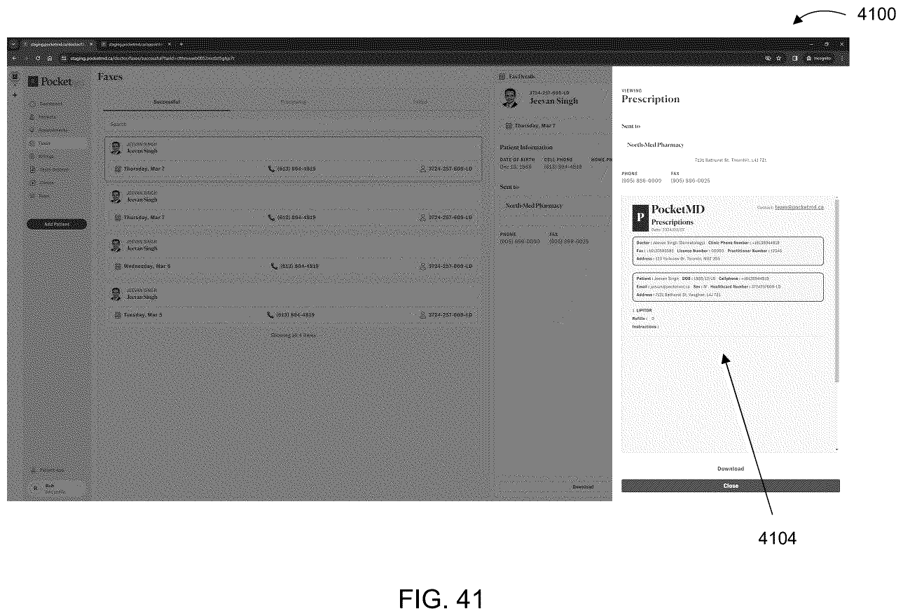

At block 610, the prescription is generated. Processor 112 may generate the prescription by querying the relevant fields within patient database 136 and physician database 140 for data that is to appear on the prescription, and then adding the medication that is prescribed by the physician. For example, when generating the prescription, the name of the patient may be queried from patient database 136, and the name and signature of the physician may be queried from physician database 140. The data is then assembled as part of a schema to create the prescription. In the current embodiment, the prescription may be generated as a PDF, however, in other embodiments, the prescription generated may be of any file type or data structure. An example of a prescription is provide at FIG. 41 below. A person skilled in the art will recognize the different fields and the locations of data that may be queried to generate the prescription.

At block 615, the prescription may be sent by processor 112 to the pharmacy via communications interface 108 and WAN 128. In the current embodiment, the prescription may be sent via electronic fax, however in alternate embodiments, the prescription may be sent through other means of electronic correspondence, such as through email or through other means into the digital inbox of a pharmacy. In yet another embodiment, the prescription may be accessed by the patient client terminal 120, where it may be printed as a hardcopy to be taken to the pharmacy by the patient. Alternatively, the prescription may remain on server 104, where it may be accessed by another client terminal (not shown) by a pharmacist. A person skilled in the art will recognize the different forms of providing the prescription to the pharmacy.

At block 620, the generated prescription is logged as part of the patient record in patient database 136. The prescription may then be accessed at any time as part of the patient's record through the graphical user interface on either the patient client terminal 120 or the physician client terminal 124.

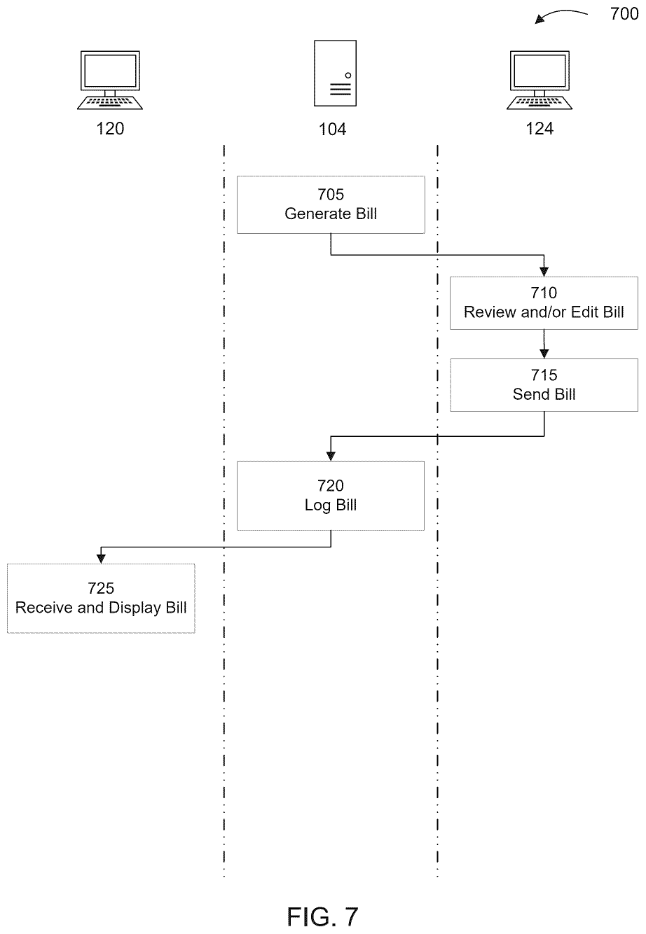

Returning to block 255, if a prescription is not needed, then method 200A and 200B may proceed to the next step of processor 112 executing the instructions provided by billing module 152 at block 700. Referring to FIG. 7, the instructions provided by the billing module 152 may include block 705, where processor 112 may generate a draft bill based on the services provided. Processor 112 may query various information from patient database 136 and physician database 140 in order to determine the line items and amount for the draft bill. In alternate embodiments, the amount and line items for the draft bill may also be determined based on the amount of time that the physician spends with the client over a virtual appointment. If an in person appointment is conducted, processor 112 may not have enough information to generate a draft bill, and hence an empty draft bill may be created for the physician to manipulate.

In alternate embodiments, a draft bill may not be created, and despite it being a virtual medical appointment, a draft bill may need to be created manually by the physician on physician client terminal 124 or server 104.

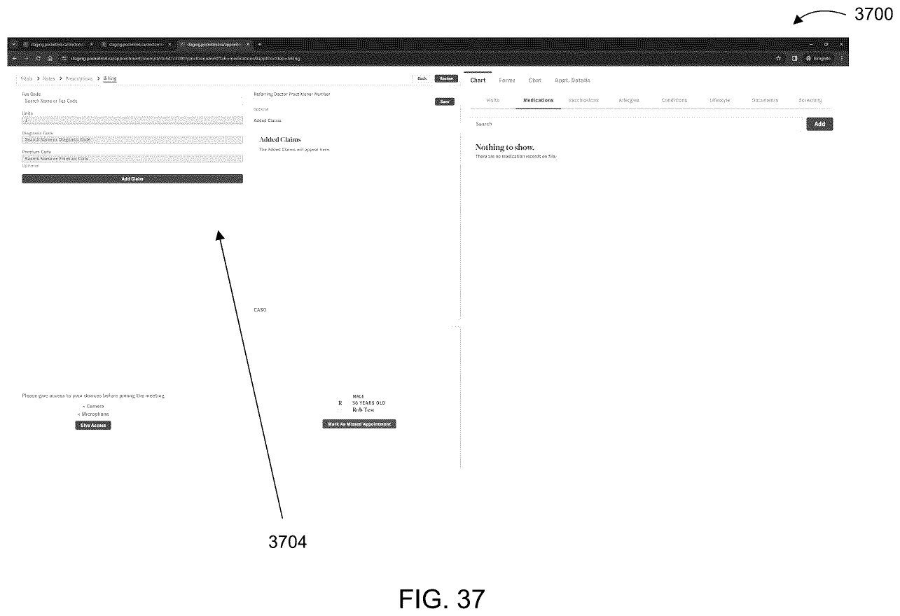

At block 710, a draft bill may be created, or an automatically generated draft bill may be reviewed and edited by the physician on physician client terminal 124 or server 104. Referring to FIG. 37, screenshot 3700 depicts an example graphical user interface where a draft bill may be reviewed and edited. For example, tab 3704 allows physicians to add or edit fee codes and claims for the bill.

Once the bill has been reviewed and finalized, it can then be sent to patient client terminal 120 to be displayed and paid by the patient. This is depicted at block 715. At block 720, the bill is logged and saved in patient database 136 and is associated with the respective patient's record. At block 725, the bill is received by patient terminal 120 and displayed for the patient.

The bill may be paid by the patient electronically via credit cards. In alternative embodiments, the patient profile may also store credit cards or other payment methods to pay bills. In yet another embodiment, if the payment is performed via credit card, an invoice may be generated and sent to patient client terminal 120 to be displayed.

In alternate embodiments, all the input of information pertaining to the vitals, prescriptions, ordering of tests, and billing may be submitted all at once, where once received, processor 112 may perform all of the relevant steps. For example, blocks 510, 610, 615, 620, 715, and 720 may all be performed after the information has been submitted. Referring to FIG. 57, screenshot 5700 displays an example graphical user interface where all the information provided by the physician, camera 156 or other sensors, may be reviewed and edited in tab 5704. Once button 5708 is clicked, processor 112 may perform all subsequent tasks. A person skilled in the art will recognize the different steps or blocks that may operate in a different order of operations, or concurrently with each other.

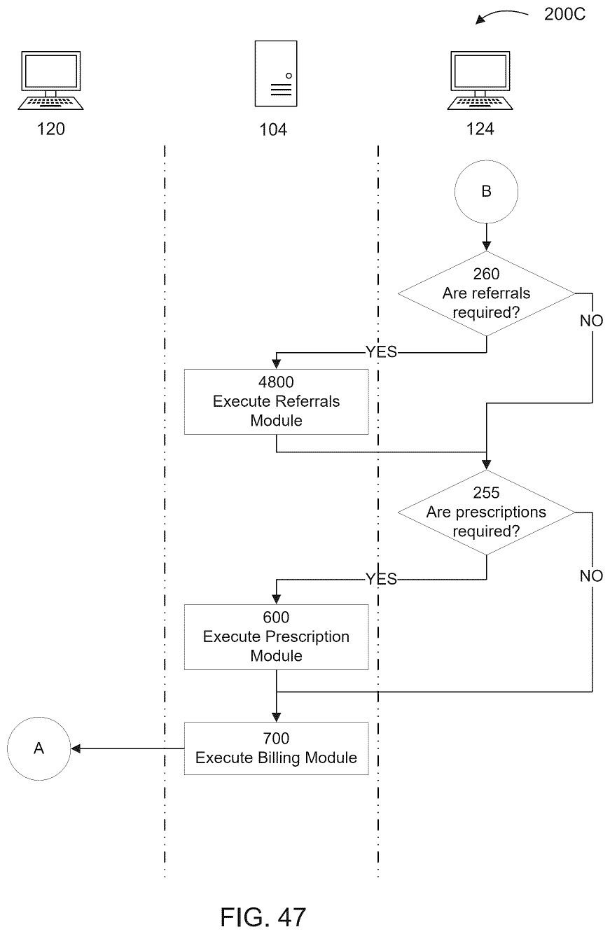

Referring to FIG. 47, in an alternate embodiment where system 100B is being used, method 200C may replace method 200B. The steps/blocks of method 200C are similar to those of method 200B, and similarly, both method 200B and 200C continue from method 200A. Hence, the steps/blocks of method 200C have similar reference numerals to those of method 200B. Method 200C differs from method 200B in that after block 250 in method 200A, where it is determined that a follow up appointment is not needed, and prior to block 255, where a physician determines whether prescriptions are required, at block 260 a physician may determine whether a referral to another medical specialist is required.

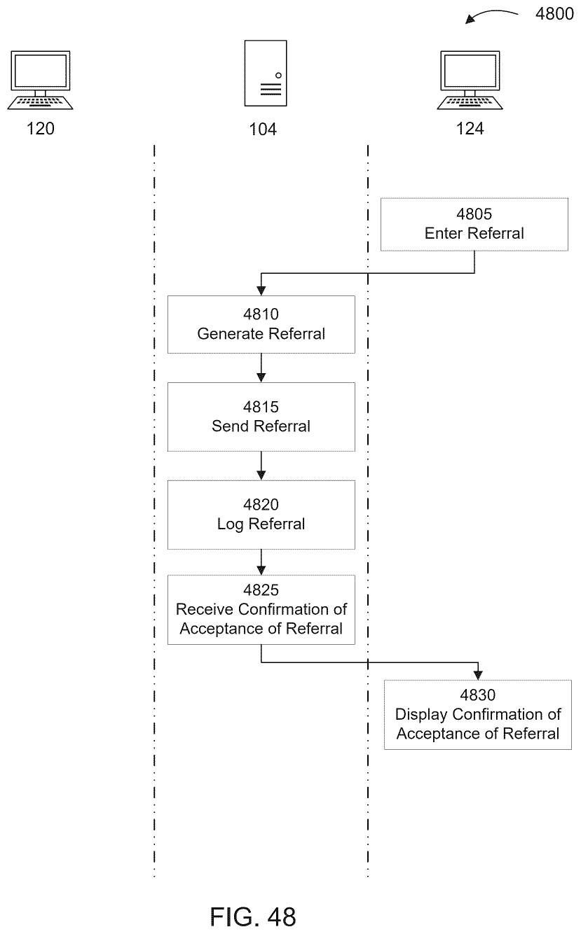

If a referral is needed, processor 112 may execute instructions provided by referrals module 160 at block 4800. Referring to FIG. 48, the instructions provided by referrals module 160 may include block 4805, where a physician may enter details regarding a request for a referral from another medical specialist.

At block 4810, the referral may be generated by processor 112, and at block 4815 the referral is sent to another medical specialist. If the medical specialist is considered a physician within physician database 140, and the medical specialist has a physician type account, the referral will appear in said medical specialists appointments. The medical specialist may then have an appointment with the patient virtually similar to the medical appointment process previously described between a physician and patient using physician client terminal 124 and patient claim terminal 120. As previously indicated, system 100, 100A, and 100B, may have more than one patient client terminal 120 and physician client terminal 124 connected to server 104. As will occur to a person skilled in the art, a medical specialist that is referred a patient may be using a second physician client terminal 124 to interact with server 104 and patient claim terminal 120.

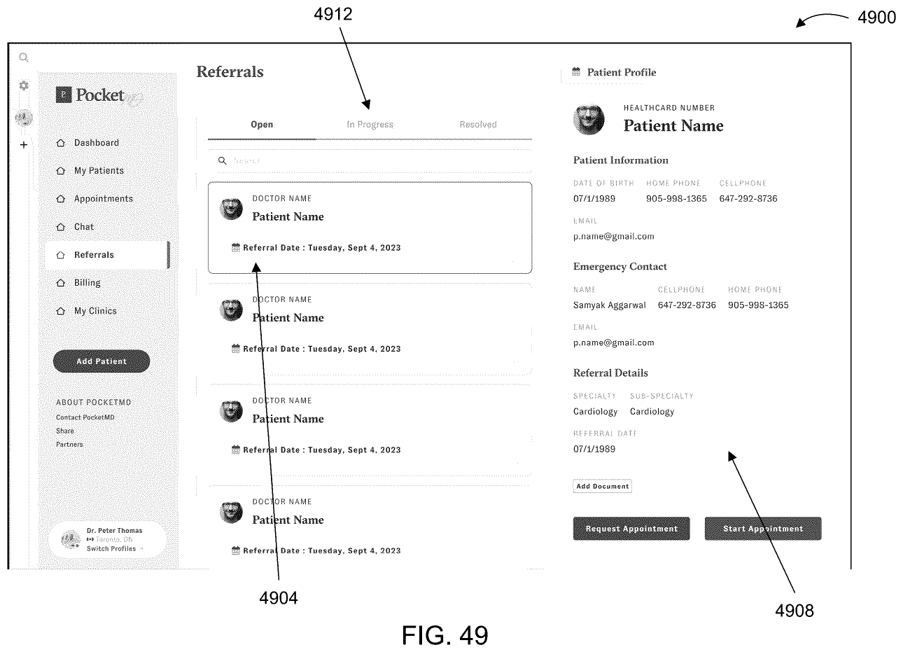

At block 4820, the referral is logged in memory 116 as part of patient database 136. Referring to FIG. 49, screenshot 4900 depicts an example graphical user interface where referrals may be listed. Specifically, tab 4904 may show a log of referrals depending on selection type. 4912. More specifically, if selection type 4912 is set to “open”, then tab 4904 lists referrals that are pending and have yet to occur. If selection type 4912 is set to “in progress”, then type 4904 lists referrals that are currently occurring as either a virtual appointment or in person appointment at the time. If selection type 4912 is set to “resolved”, then type 4904 lists a historical log of previous referrals. Tab 4908 provides details of the referral that is selected in tab 4904.

Referring to FIG. 48, block 4825 depicts a referral that has been accepted and moved from “open” status to “in progress” status, and ultimately to “resolved” status. At block 4830, the change in status is depicted and displayed on the physician client terminal 124 of the originator of the referral.

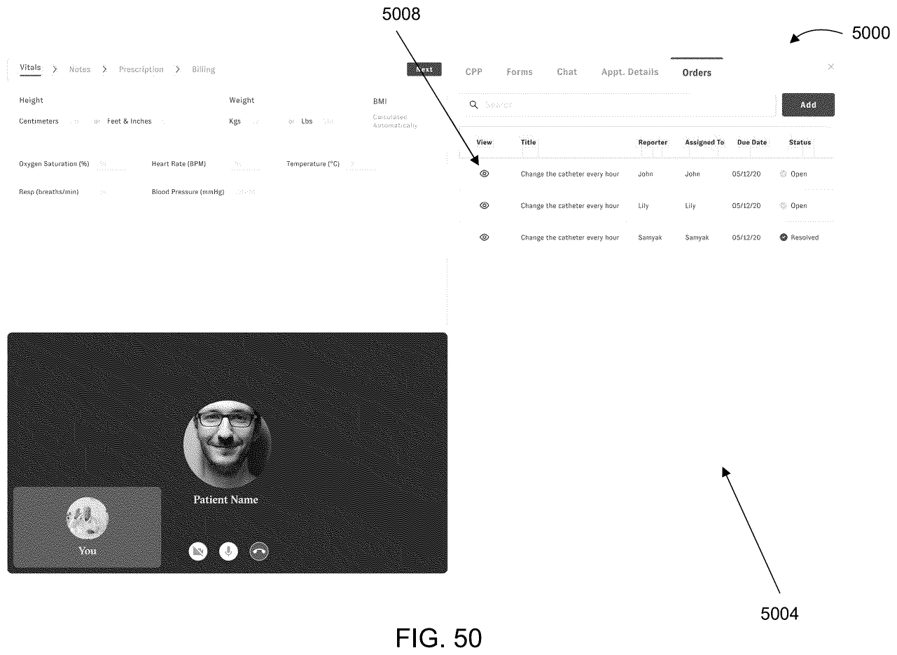

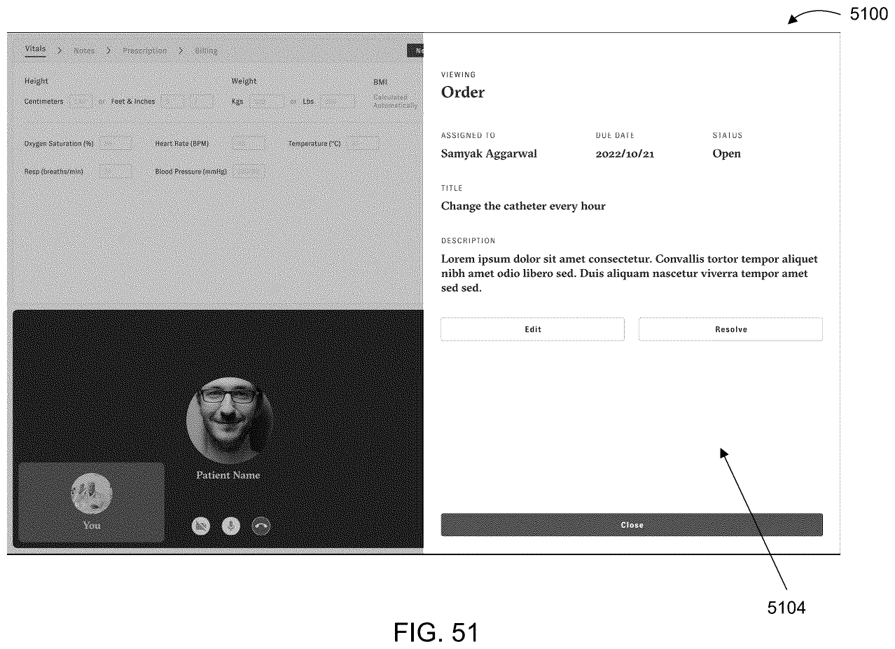

In embodiments with system 100B, or where embodiments include tasks module 164, during a medical appointment, the graphical user interface may display tasks (also referred to herein as orders) on physician client terminal 124 for the physician to complete. Specifically, tasks module 164 may provide instructions to processor 112 to provide checklist of tasks, or prompt the physician to complete a series of tasks. More specifically, memory 116 may contain a database of tasks (not shown) for multiple medical procedures that may be performed by the physician during a medical appointment. Depending on the type of medical appointment, or based on a selection of the type of medical appointment by they physician, processor 112 may query from the database of tasks, and retrieve the relevant checklist. The checklist may then be displayed on the graphical user interface. Referring to FIG. 50, screenshot 5000 depicts an example graphical user interface where tab 5004 displays a list of tasks to complete. As can be seen, tasks may also be assigned to different individuals and can be set to “complete” when the task has been performed. As such, in the event that there are multiple medical professionals involved in the appointment, either seeing the patient concurrently or one after another, tasks may be completed by each medical professional. Details regarding each task may also be provided when viewing the task at icon 5008. Referring to FIG. 51, screenshot 5100 depicts and example graphical user interface where tab 5104 displays details of the selected task.

In yet another embodiment with system 100B, or where embodiments include suggestion module 168, suggestions or alerts may be provided to the physician regarding potential actions that the physician may wish to take with respect to treating or diagnosing a patient. Suggestion module 168 may operate by instructing processor 112 to monitor statistical values from the patient, including, but not limited to, the patient's vitals, or test results. If a statistical value has surpassed a predetermined threshold, or where the statistical value is outside a certain range, then a suggestion or alert may be provided to the physician. For example, if a patient's measured blood pressure is outside a normal range, an alert may be provided to the physician to perform specific tests to determine why the blood pressure is outside the normal range. The predetermined threshold or normal ranges may be queried from a database in memory 116 (not shown). Furthermore, the predetermined thresholds or normal ranges may be based on an algorithm or other values. For example, the regular range of a heart rate may be different depending on a patient's weight, and as such, the range queried by processor 112 may be dependent on the weight of the patient. A person skilled in the art will recognize the different variables and considerations that may be used to determine the threshold or normal ranges of measurable values for a patient.

In alternative embodiments, suggestion module 168 may be an artificial intelligence engine. The artificial intelligence engine may be trained on past use cases of where multiple variables may have led to certain potential illnesses or diseases, where it may be beneficial to prompt a physician to order specific tests. Specifically, the artificial intelligence may be trained by analyzing patient's past health data and current physician notes. By thoroughly examining medical records and clinical documentation, the artificial intelligence engine of suggestion module 168 may offers valuable insights to healthcare professionals, enabling better-informed decisions about patient care and simplifying administrative tasks.

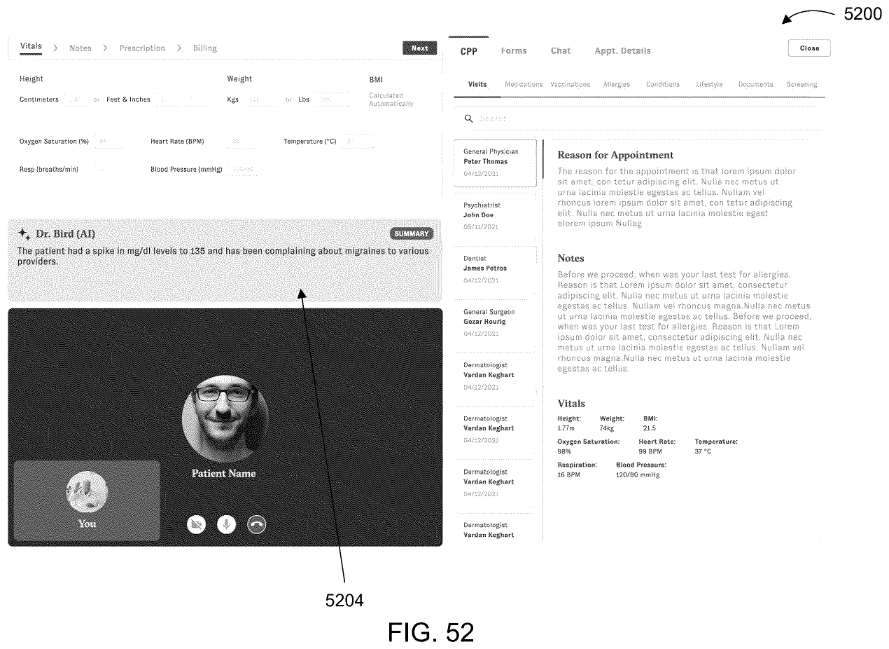

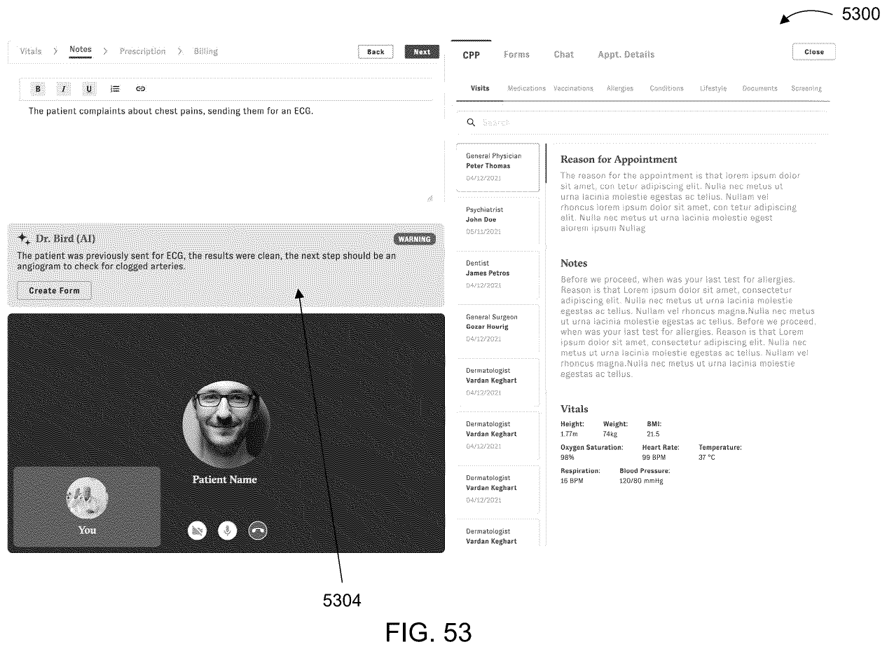

An artificial intelligence may also summarize the information provided from the notes and the medical data. Referring to FIG. 52, screenshot 5200 depicts an example graphical user interface where a summary of vitals and notes has been provided at tab 5204. Specifically, the artificial intelligence engine of suggestion module 168 summarizes the previous health history to the physician and further advises the physician of an mg/dl spike incident captured through a patient's connected sugar monitor at tab 5204.

In another example, referring to FIG. 53, screenshot 5300 depicts an example graphical user interface where a warning is provided by the artificial intelligence engine of suggestion module 168 indicating that the current diagnosis of an ECG has been completed and that a second step of an angiogram may be advised to check for clogged arteries. This is displayed at tab 5304. More specifically, in this example, symptoms from the patient may indicate an underlying issue, however, as the ECG results were clean, the artificial intelligence engine of suggestion module 168 may suggest an angiogram for further diagnosis.

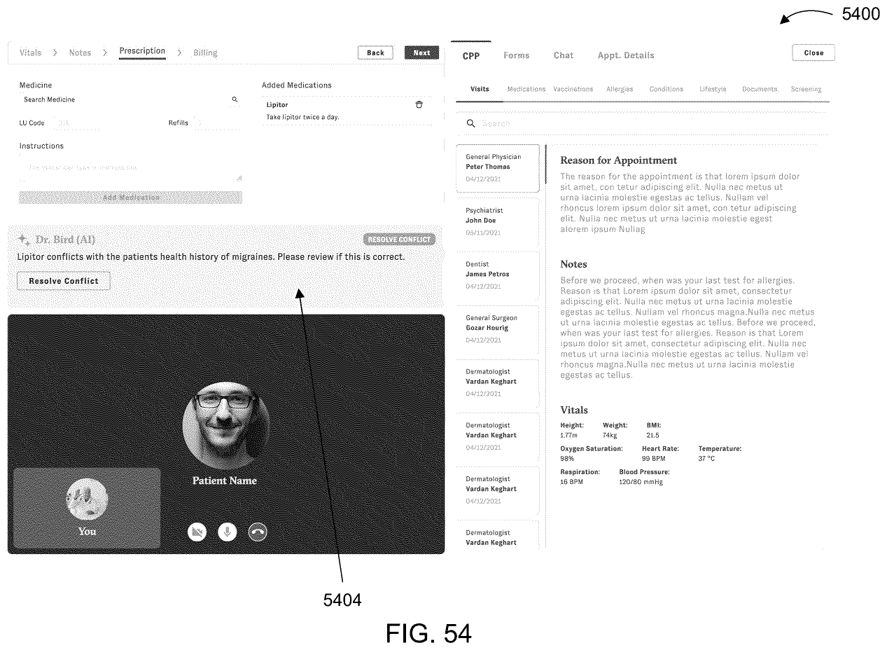

In yet another example, referring to FIG. 54, screenshot 5400 depicts an example graphical user interface where a discrepancy detected by the artificial intelligence. Engine of suggestion module 168 is displayed at tab 5404. Specifically, the artificial intelligence engine has detected that the patience health history has a pattern of migraines and that the medicine, Lipitor, may be inducing or may further induce migraines. To that end, suggestion, module 168 alerts the physician of this discrepancy and to check whether the prescribed medication is correct and safe.

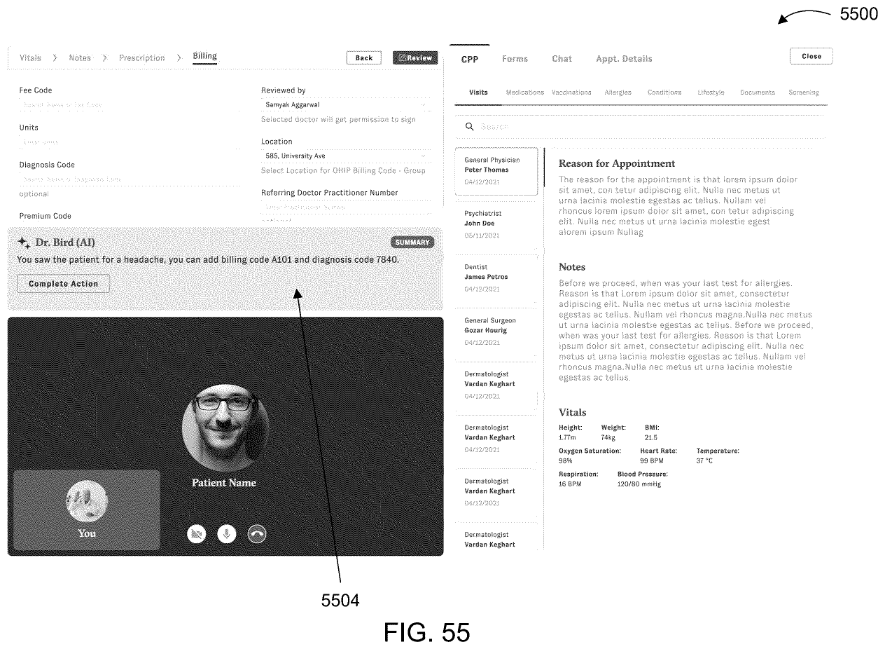

The artificial intelligence engine of suggestion module 168 is not limited to medical matters. In yet another example, referring to FIG. 55, screenshot 5500 depicts an example graphical user interface where the artificial intelligence engine of suggestion module 168 suggests at tab 5504 that certain billing codes may be used in association with the notes, tests ordered, and medication prescribed. In other embodiments, billing codes and amounts may differ depending on the patient, the patient's insurance company and the patient's insurance coverage, and the artificial intelligence engine of suggestion module 168 may determine the best billing cods and pricing breakdowns to use.

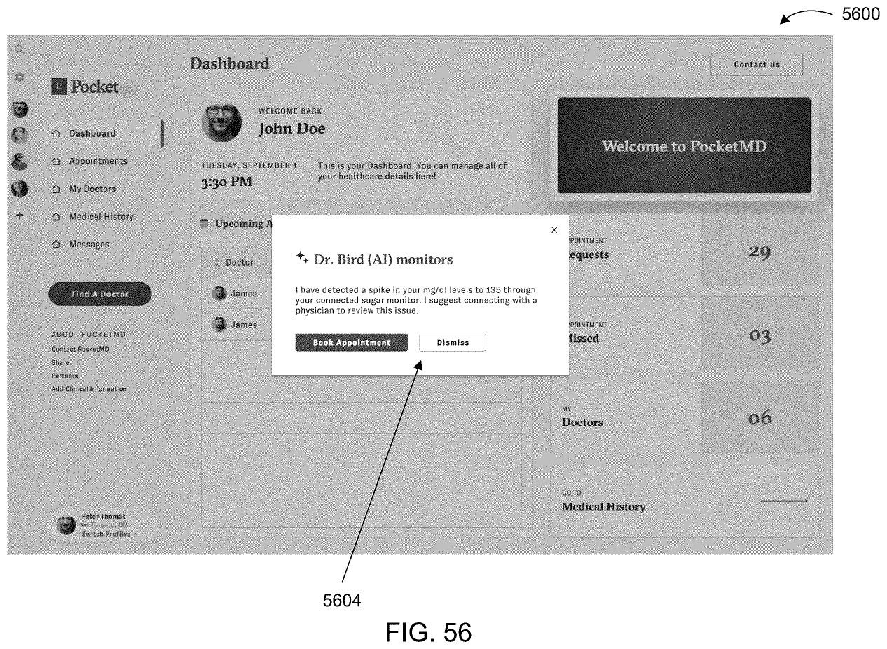

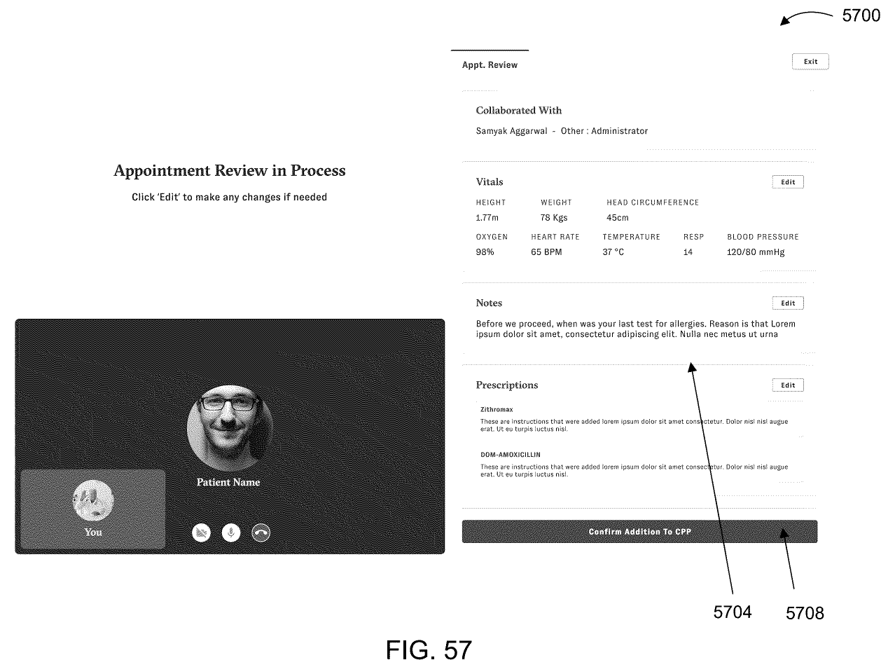

Suggestion module 168 is not limited to providing suggestions or alerts to physicians, but may also provide suggestions or alerts to patients as well. Referring to FIG. 56, screenshot 5600 depicts an example graphical user interface on patient client terminal 120 where alert 5604 is shown indicating that a connected sugar monitor has detected a spike in mg/dl levels. Suggestion module 168 may then advise the patient to schedule a medical appointment with a physician based on the detected discrepancy. A person skilled in the art will recognize that any sensor or any values collected may be analyzed by the artificial intelligence engine of suggestion module 168 to provide recommendations for alerts.

The graphical user interface also provides for patients and physicians to access the patient's medical history and data from patient database 136. By providing the patient's medical history and data in one centralized location, it can be updated and viewed by multiple physicians, allowing for additional flexibility for patients to schedule appointments with multiple physicians. Alternatively, if the patient relocates to a new city where the physicians are different, the patient's medical history may be accessed easily by the new physicians.

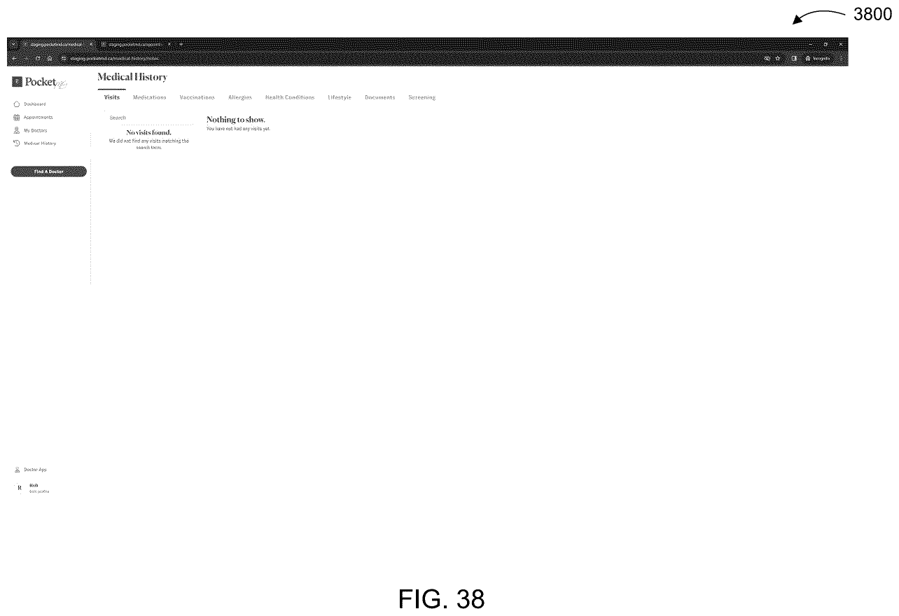

Referring to FIG. 38, screenshot 3800 depicts an example graphical user interface that a patient may see when accessing patient's medical history from patient client terminal 120. Specifically, the patient may see example screenshot 3800 when accessing the patient's medical history from a patient account. As can be seen in screenshot 3800, the past appointments/visits, medications, vaccinations, allergies, health conditions, lifestyle activities, documents and screenings can be seen.

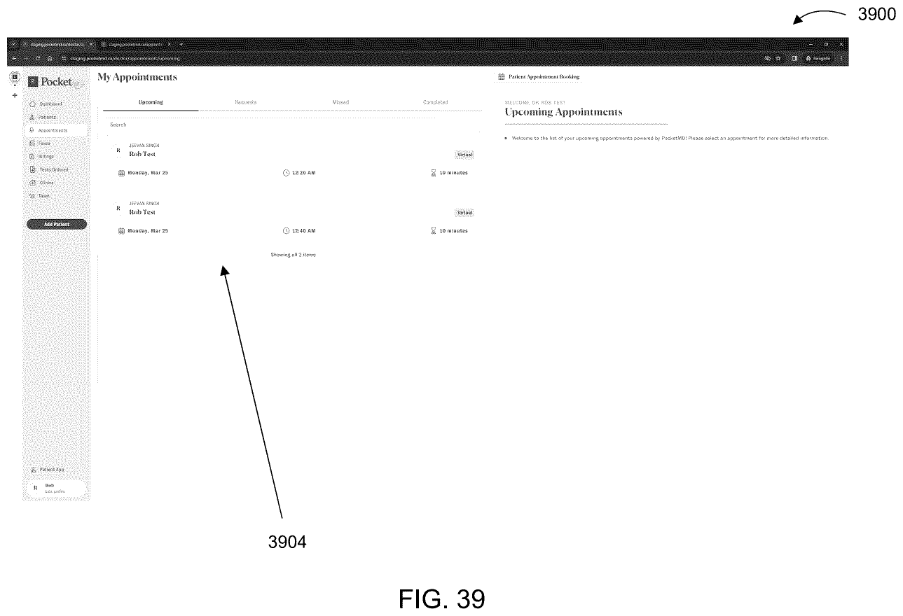

Physician data is also accessible on server 104. Referring to FIG. 39, screenshot 3900 depicts an example graphical user interface that a physician may see when accessing a physicians appointments from physician client terminal 124. Specifically, 3900 at tab 3904 the upcoming appointments, request for appointments, missed appointments, and completed appointments for the physician are shown.

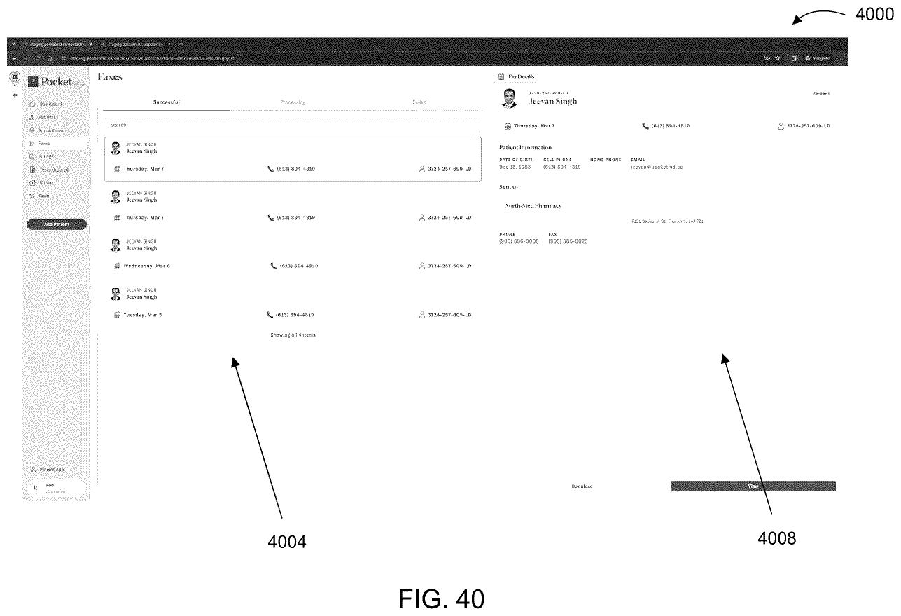

Referring to FIG. 40, screenshot 4000 depicts an example graphical user interface of faxes that processor 112 may have sent on behalf of the physician. Examples of faxes may include prescriptions that are sent to pharmacies, tests that are sent to labs or other medical professionals, and/or bills. In the current embodiment the faxes shown on screenshot 4000 are prescriptions sent to pharmacies. Tab 4004 shows a list of successful faxes that were sent. Tab 4004 may also show faxes that are processing or failed. Faxes. Tab 4008 provides details of the fax that is selected in tab 4004. Referring to FIG. 41, screenshot 4100 depicts an example graphical user interface of a prescription that was sent via fax in tab 4104. The physician may review the fax that was previously generated and sent. A person skilled in the art will recognize the different documents and/or faxes that may be shown as part of the graphical user interface.

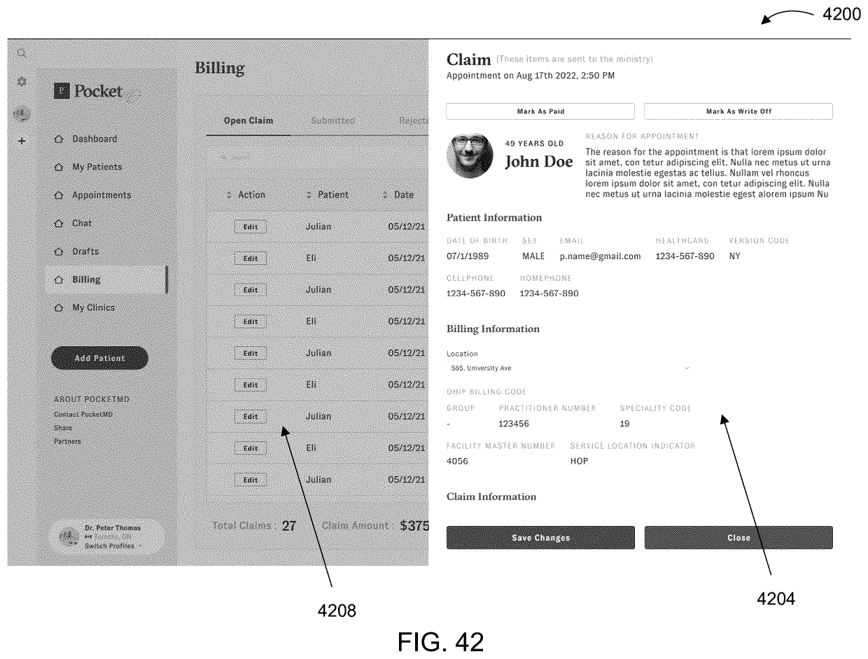

Referring to FIG. 42, screenshot 4200 depicts an example graphical user interface displaying a series of claims either pending or in the past at tab 4208 and a claim or a bill that was previously sent out at tab 4204. As previously provided, bills and claims may be sent to the patient. However, in other embodiments such as the one displayed in screenshot 4200, the claim at tab 4204 was sent to the ministry. In alternative embodiments, claims may also be sent to insurance providers. In yet another alternative embodiment, bills may also have different statuses. For example, bills or claims may be considered either “open” if they are pending review, “submitted” if the claims have been submitted to an insurance company, “approved” if the claims have been approved by the insurance company, or “denied” if the claims have been denied by the insurance company. A list of claims based on the status may also be displayed in tab 4208. Claims may also be added in tab 4204. A person skilled in the art will recognize the different functions and destinations for claims and bills. A person skilled in the art will also recognize the change in layout of the graphical user interface and the fields depending on the type of claim or bill.

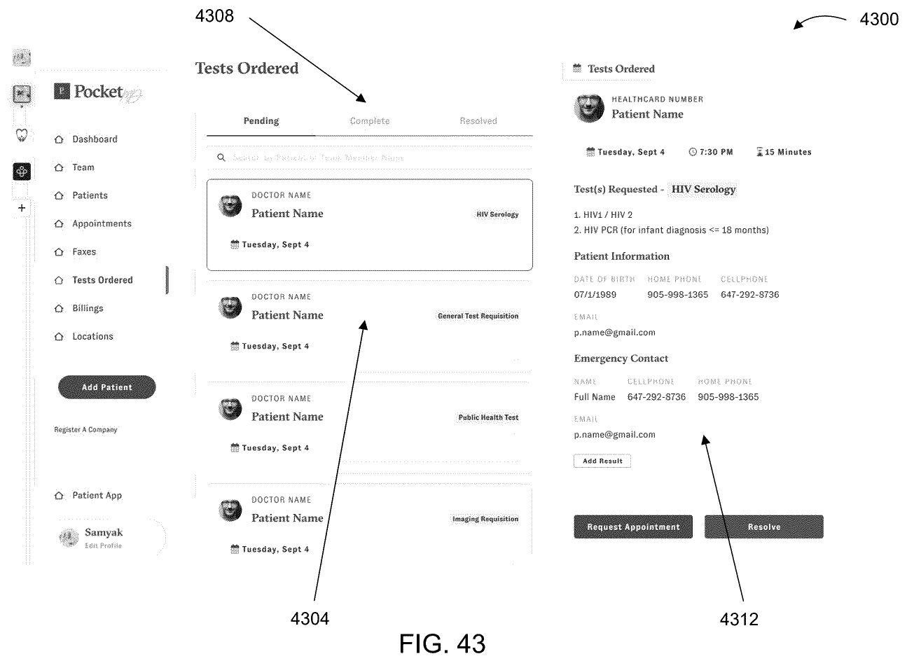

Referring to FIG. 43, screenshot 4300 depicts an example graphical user interface showing pending medical tests or procedures ordered and a log of the results of all medical test or procedures ordered by the physician of a specific physician account. Specifically, selection type 4308 toggles the lists of tests ordered displayed at tab 4304. More specifically, selection type 4308 toggles between the “pending” status, “complete” status” and “resolved” status for tests ordered. Tests ordered that are considered “pending” status are tests that have yet to be completed by the lab. Tests ordered that are considered “complete” are where the medical test results have been received by server 104 and the medical test results are in patient database 136. Tests that are considered “resolved” are where the physician has reviewed the medical test results and has either requested a follow up medical appointment, as is provided at block 250 at FIG. 2, or where no follow up action is required. When a test is selected in tab 4304, the details of the test may be provided in tab 4312.

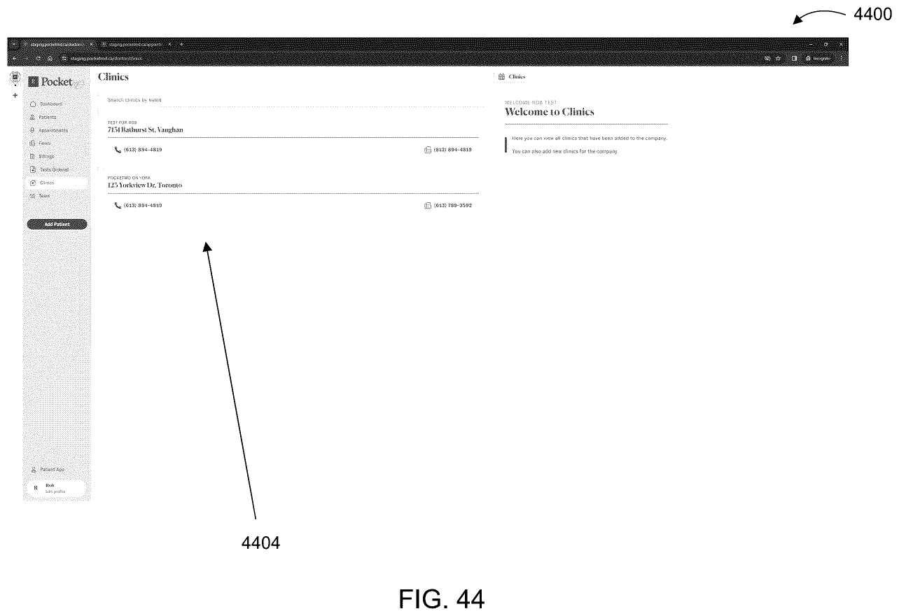

Referring to FIG. 44, screenshot 4400 depicts an example graphical user interface of all the clinics associated with the physician of a specific physician account. A physician may work at multiple clinics, and as such by providing said clinics at tab 4404, patients may select which clinic they wish to visit when scheduling an appointment with the physician.

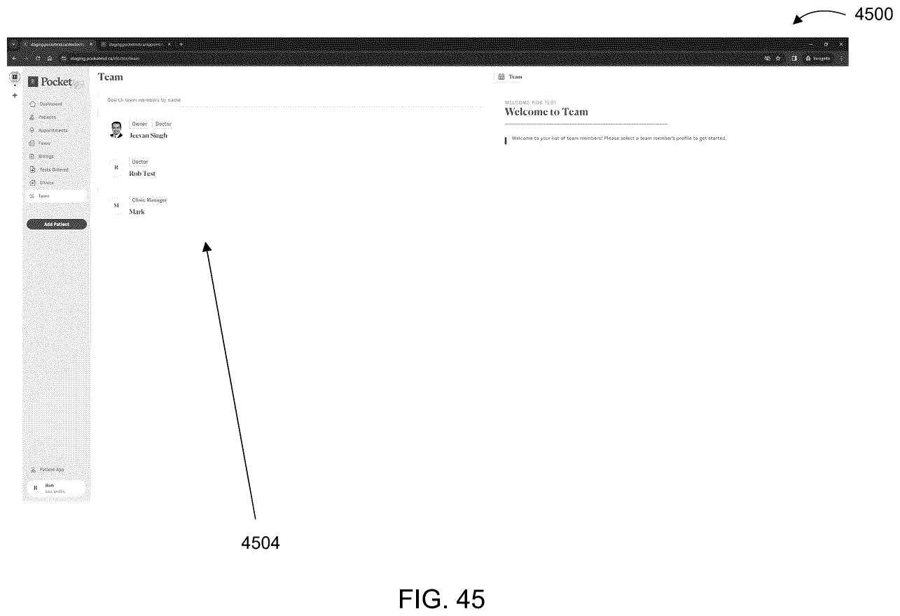

Referring to FIG. 45, screenshot 4500 depicts an example graphical user interface of the team associated with The physician of a specific physician account. A physician may be part of a larger team of other medical professionals. The team is not limited to other physicians but may include other roles including, but not limited to, clinic manager, nurses, and therapists.

Examples of graphical user interfaces are described and provided as figures throughout. A person skilled in the art will recognize that different layouts for the graphical user interfaces are contemplated.

Although the foregoing description and accompanying drawings to specific preferred embodiments of the present invention as presently contemplated by the inventor, it will be understood that various changes, modifications and adaptations, may be made without departing from the spirit of the invention.

The embodiments for which an exclusive privilege or property is claimed are as follows:

1. A system for managing a medical practice, the system comprising:
a communications interface connected to a network, wherein the communications interface is configured to receive data from a first client terminal and a second client terminal;
a memory storage unit including:
a database of patient medical records;
a processor connected to the communications interface and the memory storage unit, the processor configured to:
schedule patient appointments requested from the first client terminal;
receive medical data from a second client terminal;
store the medical data in the database of medical records;
if a medical test is needed based on the received medical data:
generate a request for the medical test;
send the request for the medical test to a medical test provider; and
receive a test result from the medical test provider;
if a prescription is needed based on the received medical data:
generate the prescription; and
send the prescription electronically to a pharmacy.

2. The system of claim 1, wherein the processor is further configured to generate an insurance claim, and send the insurance claim to a medical insurance provider.

3. The system of claim 1, where the memory storage unit further includes an artificial intelligence engine trained on the database of medical records, the processor further configured to provide suggestions of whether a medical test or prescription is needed based the received medical data using the artificial intelligence engine.

4. The system of claim 1, where the memory storage unit further includes a database of physician information, wherein the processor configured to generate the prescription includes:
receiving a medication to be prescribed from the second client terminal;
querying the database of medical records to retrieve the patient's name;
querying the database of physician information to retrieve a physician's name and a physician's signature; and
assembling the prescription, the prescription including at least the medication to be prescribed, the patient's name, the physician's name and the physician's signature.

5. The system of claim 1, wherein the communications interface is further configured to receive data from a validation server, where the database of patient medical records includes patient insurance data, wherein the processor is further configured to verify the patient insurance data against the validation server prior scheduling patient appointments requested from the first client terminal.

6. A method of administering a medical appointment, the method comprising:
scheduling the medical appointment from a first client terminal;
receiving patient medical data from a second client terminal;
storing the received patient medical data in a database of medical records;
determining whether a medical test is needed based on the received medical data, where if the medical test is needed:
generating a request for the medical test;
sending the request for the medical test to the first client terminal;
receiving a test result from a medical test provider;
determining whether a prescription is needed based on the received medical data, where if the prescription is needed:
generating the prescription; and
sending the prescription electronically to a pharmacy.

7. The method of claim 6 further comprising generating an insurance claim and sending the insurance claim to a medical insurance provider.

8. The method of claim 6 further comprising providing suggestions of whether the medical test or prescription is needed based on the received medical data using an artificial intelligence engine trained on the database of medical records.

9. The method of claim 6, wherein generating the prescription further includes:
receiving a medication to be prescribed from the second client terminal;
querying the database of medical records to retrieve the patient's name;
querying the database of physician information to retrieve a physician's name and a physician's signature; and
assembling the prescription, the prescription including at least the medication to be prescribed, the patient's name, the physician's name and the physician's signature.

10. The method of claim 6 further comprising receiving patient insurance data from the first client terminal, and verifying the patient insurance data against a validation server prior to scheduling patient appointments requested from the first client terminal.
